SEQUENCE LISTING (18 sequences)

SEQ ID NO: 1 - DNA, 645 bp, Bos taurus
  source 1..645: mol_type = other DNA
atgaaacttctcatccttacctgtcttgtggctgttgctcttgccaggcctaaacatcctatcaagcaccaaggactccctcaagaagtcctcaatgaaaatttactcaggttttttgtggcaccttttccagaagtgtttggaaaggagaaggtcaatgaactgagcaaggatattgggagtgaatcaactgaggatcaagccatggaagatattaagcaaatggaagctgaaagcatttcgtcaagtgaggaaattgttcccaatagtgttgagcagaagcacattcaaaaggaagatgtgccctctgagcgttacctgggttatctggaacagcttctcagactgaaaaaatacaaagtaccccagctggaaattgttcccaatagtgctgaggaacgacttcacagtatgaaagagggaatccatgcccaacagaaagaacctatgataggagtgaatcaggaactggcctacttctaccctgagcttttcagacaattctaccagctggatgcctatccatctggtgcctggtattacgttccactaggcacacaatacactgatgccccatcattctctgacatccctaatcccattggctctgagaacagtgaaaagactactatgccactgtggtga

SEQ ID NO: 2 - DNA, 645 bp, synthetic construct
  source 1..645: mol_type = other DNA
atgaagctgctgatcctgacctgcctcgtggctgtagccctcgctcgccctaaacatccaattaaacatcagggtttgccacaagaggttctgaatgagaatctcctgcgttttttcgttgctccattcccagaagttttcggcaaagaaaaagttaacgaactttcaaaagatatcggttcagagagtacagaggaccaagcaatggaagatataaaacaaatggaggctgagtctattagtagctccgaggaaatagtgcctaattccgttgaacagaaacacatccagaaggaagatgtccctagtgagagatacttgggttacttggaacagttgttgcgtttgaaaaagtacaaggtccctcaattggagatagttcctaatagcgccgaggagcgtttgcactctatgaaggagggcatacatgcccaacaaaaagaacctatgatcggcgtgaaccaggagctggcttacttttaccctgaactttttcgtcagttctaccagttggacgcctatccctccggcgcatggtactatgtacccctgggtacacaatataccgacgcaccctcatttagtgatatacctaatcccatcggatcagaaaatagtgaaaagaccactatgccactttggtga

SEQ ID NO: 3 - DNA, 669 bp, Bos taurus
  source 1..669: mol_type = other DNA
atgaagttcttcatctttacctgccttttggctgttgcccttgcaaagaatacgatggaacatgtctcctccagtgaggaatctatcatctcccaggaaacatataagcaggaaaagaatatggacattaatcccagcaaggagaacctttgctccacattctgcaaggaagttgtaaggaacgcaaatgaagaggaatattctatcggctcatctagtgaggaatctgctgaagttgccacagaggaagttaagattactgtggacgataagcactaccagaaagcactgaatgaaatcaatcagttttatcggaagttcccccagtatctccagtatctgtatcaaggtccaattgttttgaacccatgggatcaggttaagagaaatgctgttcccattactcccactctgaacagagagcagctctccaccagtgaggaaaattcaaagaagaccgttgacatggaatcaacagaagtattcactaagaaaactaaactgactgaagaagaaaagaatcgcctaaattttctgaaaaaaatcagccagcgttaccagaaattcgccttgccccagtatctcaaaactgtttatcagcatcagaaagctatgaagccatggattcaacctaagacaaaggttattccctatgtgaggtacctttaa

SEQ ID NO: 4 - DNA, 669 bp, synthetic construct
  source 1..669: mol_type = other DNA
atgaagttcttcatattcacctgccttcttgccgtagctttggcaaagaacacaatggagcatgtgagttcaagtgaagaatccattatcagccaagaaacctacaaacaagagaagaatatggacataaacccctcaaaggaaaacctgtgttctacattttgcaaagaggtggttcgtaatgcaaatgaggaagagtattccatcggctcaagctcagaagagagtgcagaggttgctaccgaagaggttaagataacagtggatgataagcactatcaaaaagccctgaatgaaattaaccagttctaccgtaagttccctcaatatcttcagtatctgtaccaaggtcccattgtccttaatccatgggatcaggtcaaaaggaatgccgtgcccataactcctacacttaacagagaacaactgtctaccagcgaagagaacagcaagaaaaccgtcgatatggaatctaccgaggttttcaccaagaaaaccaaattgacagaggaagaaaagaataggctcaattttctcaagaaaatttctcagcgctatcaaaaattcgcacttccacaatacctcaagacagtgtaccagcaccaaaaagccatgaaaccatggattcaacccaaaacaaaggttatcccctatgtcagatacctgtaa

SEQ ID NO: 5 - DNA, 675 bp, Bos taurus
  source 1..675: mol_type = other DNA
atgaaggtcctcatccttgcctgcctggtggctctggcccttgcaagagagctggaagaactcaatgtacctggtgagattgtggaaagcctttcaagcagtgaggaatctattacacgcatcaataagaaaattgagaagtttcagagtgaggaacagcagcaaacagaggatgaactccaggataaaatccacccctttgcccagacacagtctctagtctatcccttccctgggcccatccataacagcctcccacaaaacatccctcctcttactcaaacccctgtggtggtgccgcctttccttcagcctgaagtaatgggagtctccaaagtgaaggaggctatggctcctaagcacaaagaaatgcccttccctaaatatccagttgagccctttactgaaaggcagagcctgactctcactgatgttgaaaatctgcaccttcctctgcctctgctccagtcttggatgcaccagcctcaccagcctcttcctccaactgtcatgtttcctcctcagtccgtgctgtccctttctcagtccaaagtcctgcctgttccccagaaagcagtgccctatccccagagagatatgcccattcaggcctttctgctgtaccaggagcctgtactcggtcctgtccggggacccttccctattattgtctaa

SEQ ID NO: 6 - DNA, 675 bp, synthetic construct
  source 1..675: mol_type = other DNA
atgaaagtcctgatactggcttgccttgtcgccctggcacttgcccgcgaactggaggagctcaatgtacctggcgaaatagtggagagtctctcaagttccgaagagagcataacaagaatcaacaaaaagatcgagaaatttcaatcagaggaacagcaacagactgaggatgaactccaagacaaaatccatccatttgcccagactcagtcattggtctatccctttccaggccccatccacaactccctgccacagaatatccctccattgacacagacccccgttgtggtgcctccattccttcaaccagaggtcatgggagtgagtaaggtaaaggaagcaatggctccaaaacacaaggaaatgccatttcccaaatacccagtcgagcctttcacagaaaggcagtctcttaccctgactgatgtggaaaacttgcacttgcccctgcccctcttgcaatcatggatgcaccagccccaccagcccctgcctcctaccgttatgtttcctccacagagcgtactgtccttgagccagtcaaaagtcctccctgtgcctcaaaaagctgttccttatccccagcgtgacatgcctatccaagcatttctcctctatcaggagccagtgctgggacctgtgcgtggtcccttcccaataatagtctaa

SEQ ID NO: 7 - DNA, 573 bp, Bos taurus
  source 1..573: mol_type = other DNA
atgatgaagagttttttcctagttgtgactatcctggcattaaccctgccatttttgggtgcccaggagcaaaaccaagaacaaccaatacgctgtgagaaagatgaaagattcttcagtgacaaaatagccaaatatatcccaattcagtatgtgctgagtaggtatcctagttatggactcaattactaccaacagaaaccagttgcactaattaataatcaatttctgccatacccatattatgcaaagccagctgcagttaggtcacctgcccaaattcttcaatggcaagttttgtcaaatactgtgcctgccaagtcctgccaagcccagccaactaccatggcacgtcacccacacccacatttatcatttatggccattccaccaaagaaaaatcaggataaaacagaaatccctaccatcaataccattgctagtggtgagcctacaagtacacctaccatcgaagcagtagagagcactgtagctactctagaagcttctccagaagttattgagagcccacctgagatcaacacagtccaagttacttcaactgcggtctaa

SEQ ID NO: 8 - DNA, 573 bp, synthetic construct
  source 1..573: mol_type = other DNA
atgatgaagtcattctttctggtagtcactatattggctctgacattgccctttctcggtgctcaagagcagaaccaggaacaacccatcaggtgtgaaaaagatgaaagattcttttctgataagattgccaagtacatacccattcagtatgttctttcacgttatccatcatatggcctcaactattatcagcagaagccagtcgcccttattaataaccaattccttccctacccctattacgctaaacctgctgctgtccgctctcccgcccaaatactccagtggcaagttctcagtaatactgtgcccgccaagtcctgtcaagctcagcccacaacaatggcacgtcacccacatccacacctcagctttatggcaattccaccaaagaaaaatcaagataaaaccgagatccctaccatcaacactattgcttccggtgagcctacctctacacctactatcgaggcagtagagagtacagtcgcaactcttgaggcatcaccagaggtcatcgagagtccccccgaaataaacaccgttcaagtgaccagcactgccgtataa

SEQ ID NO: 9 - protein, 214 aa, Bos taurus
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 10 - protein, 222 aa, Bos taurus
MKFFIFTCLLAVALAKNTMEHVSSSEESIISQETYKQEKNMDINPSKENLCSTFCKEVVRNANEEEYSIGSSSEESAEVATEEVKITVDDKHYQKALNEINQFYRKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 11 - protein, 224 aa, Bos taurus
MKVLILACLVALALARELEELNVPGEIVESLSSSEESITRINKKIEKFQSEEQQQTEDELQDKIHPFAQTQSLVYPFPGPIHNSLPQNIPPLTQTPVVVPPFLQPEVMGVSKVKEAMAPKHKEMPFPKYPVEPFTERQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 12 - protein, 190 aa, Bos taurus
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTIEAVESTVATLEASPEVIESPPEINTVQVTSTAV

SEQ ID NO: 13 - DNA, 978 bp, Bos taurus
  source 1..978: mol_type = other DNA
atggccagcagcagcggcagcaaggccgagttcatcgtgggcggcaagtacaagctggtgaggaagatcggcagcggcagcttcggcgacatctacctggccatcaacatcaccaacggcgaggaggtggccgtgaagctggagagccagaaggccaggcacccccagctgctgtacgagagcaagctgtacaagatcctgcagggcggcgtgggcatcccccacatcaggtggtacggccaggagaaggactacaacgtgctggtgatggacctgctgggccccagcctggaggacctgttcaacttctgcagcaggaggttcaccatgaagaccgtgctgatgctggccgaccagatgatcagcaggatcgagtacgtgcacaccaagaacttcatccacagggacatcaagcccgacaacttcctgatgggcatcggcaggcactgcaacaagctgttcctgatcgacttcggcctggccaagaagtacagggacaacaggaccaggcagcacatcccctacagggaggacaagaacctgaccggcaccgccaggtacgccagcatcaacgcccacctgggcatcgagcagagcaggagggacgacatggagagcctgggctacgtgctgatgtacttcaacaggaccagcctgccctggcagggcctgaaggccgccaccaagaagcagaagtacgagaagatcagcgagaagaagatgagcacccccgtggaggtgctgtgcaagggcttccccgccgagttcgccatgtacctgaactactgcaggggcctgaggttcgaggaggcccccgactacatgtacctgaggcagctgttcaggatcctgttcaggaccctgaaccaccagtacgactacaccttcgactggaccatgctgaagcagaaggccgcccagcaggccgccagcagcagcggccagggccagcaggcccagacccccaccggcttctaa

SEQ ID NO: 14 - DNA, 978 bp, synthetic construct
  source 1..978: mol_type = other DNA
atggcttctagtagtgggagtaaggcagaattcatcgtaggcgggaaatacaaacttgtacgcaagatcggttctgggtctttcggcgatatctacctcgccatcaacatcaccaatggcgaggaggtagccgtaaaacttgaatcacagaaggctagacatccacagctcctctacgagagtaaactctataaaatactgcaaggaggagttggaatcccacatattaggtggtacggccaggagaaagattacaatgtcctcgtgatggatctgcttggccccagcttggaggatcttttcaatttttgttccagacgcttcaccatgaaaactgttcttatgctggccgatcaaatgatttcacgtattgagtatgtacacaccaagaacttcatccatcgtgacatcaaaccagacaattttctcatggggattgggagacattgcaacaaacttttccttatcgattttggtttggcaaaaaaatacagggacaaccgcacacgtcagcatataccctatagagaggacaagaacttgaccggcaccgcacgctatgcctctataaatgctcatttgggaatcgaacaaagtagacgtgacgacatggaatccttgggctatgttcttatgtacttcaaccgtactagccttccatggcagggcctcaaggcagcaacaaagaaacagaagtacgaaaaaatctctgaaaagaaaatgagcacacctgtagaggttctttgtaaaggttttcccgctgaattcgctatgtatcttaattactgtcgcggactccgcttcgaggaagcacctgactatatgtatttgcgtcagctgtttagaattctgtttagaacactcaaccatcaatatgactatacattcgactggacaatgctcaagcaaaaagccgcacagcaggctgcttcatcctccggtcagggacagcaagcccaaactccaactggcttttaa

SEQ ID NO: 15 - DNA, 1176 bp, Bos taurus
  source 1..1176: mol_type = other DNA
atgagcggccccgtgcccagcagggccagggtgtacaccgacgtgaacacccacaggcccagggagtactgggactacgagagccacgtggtggagtggggcaaccaggacgactaccagctggtgaggaagctgggcaggggcaagtacagcgaggtgttcgaggccatcaacatcaccaacaacgagaaggtggtggtgaagatcctgaagcccgtgaagaagaagaagatcaagagggagatcaagatcctggagaacctgaggggcggccccaacatcatcaccctggccgacatcgtgaaggaccccgtgagcaggacccccgccctggtgttcgagcacgtgaacaacaccgacttcaagcagctgtaccagaccctgaccgactacgacatcaggttctacatgtacgagatcctgaaggccctggactactgccacagcatgggcatcatgcacagggacgtgaagccccacaacgtgatgatcgaccacgagcacaggaagctgaggctgatcgactggggcctggccgagttctaccaccccggccaggagtacaacgtgagggtggccagcaggtacttcaagggccccgagctgctggtggactaccagatgtacgactacagcctggacatgtggagcctgggctgcatgctggccagcatgatcttcaggaaggagcccttcttccacggccacgacaactacgaccagctggtgaggatcgccaaggtgctgggcaccgaggacctgtacgactacatcgacaagtacaacatcgagctggaccccaggttcaacgacatcctgggcaggcacagcaggaagaggtgggagaggttcgtgcacagcgagaaccagcacctggtgagccccgaggccctggacttcctggacaagctgctgaggtacgaccaccagagcaggctgaccgccagggaggccatggagcacccctacttctacaccgtggtgaaggaccaggccaggatgggcagcagcagcatgcccggcggcagcacccccgtgagcagcgccaacatgatgagcggcatcagcagcgtgcccacccccagccccctgggccccctggccggcagccccgtgatcgccgccgccaaccccctgggcatgagcgtgcccgccgccgccggcgcccagcagtaa

SEQ ID NO: 16 - DNA, 1176 bp, synthetic construct
  source 1..1176: mol_type = other DNA
atgtccggccccgttccatcccgcgccagggtctatactgatgtgaatacccataggcccagggaatattgggattatgagtcacacgtagtggagtggggtaaccaagacgattatcaactcgtgaggaaactcggcaggggcaaatacagtgaggtgttcgaggctatcaatataaccaataatgaaaaggttgttgtgaagatcctgaaaccagttaagaaaaagaaaatcaaaagggaaataaaaatccttgaaaatttgcgtggtggaccaaatatcattacacttgctgacatagtgaaggatccagtttcacgcactcccgcattggtatttgagcacgttaataacaccgacttcaaacagctttaccaaaccctcaccgattatgatatacgcttctacatgtatgagatattgaaagccctggattattgtcacagtatgggtatcatgcaccgcgacgtaaagccccacaatgttatgattgaccacgagcatcgtaagctcagacttatagattggggactcgctgagttttaccatcccggacaggagtataatgtgcgcgtcgcaagtcgttatttcaaaggtcctgaactgttggtcgattatcaaatgtacgattactctcttgatatgtggtctttgggatgcatgctggcttcaatgatatttaggaaggagcctttttttcacggacatgacaattatgaccaattggtgagaatagccaaagtgcttggaactgaagatctctatgactacattgacaaatacaacattgagctggatcccagattcaacgacatcctgggaaggcactcaagaaagagatgggagaggtttgtccactctgagaaccagcatctcgtaagccctgaagctctcgatttcctggataaattgcttcgttacgatcatcagtcacgtctcactgctcgcgaggctatggaacatccttatttttatactgtcgtaaaggaccaggcccgcatgggtagctccagtatgcccggaggcagtactccagtaagcagtgccaatatgatgtcaggaataagctctgttccaacccccagtcctttggggccactcgccggatctccagtaattgcagccgctaaccctctgggaatgagcgttcccgctgcagcaggggcccaacagtaa

SEQ ID NO: 17 - protein, 325 aa, Bos taurus
MASSSGSKAEFIVGGKYKLVRKIGSGSFGDIYLAINITNGEEVAVKLESQKARHPQLLYESKLYKILQGGVGIPHIRWYGQEKDYNVLVMDLLGPSLEDLFNFCSRRFTMKTVLMLADQMISRIEYVHTKNFIHRDIKPDNFLMGIGRHCNKLFLIDFGLAKKYRDNRTRQHIPYREDKNLTGTARYASINAHLGIEQSRRDDMESLGYVLMYFNRTSLPWQGLKAATKKQKYEKISEKKMSTPVEVLCKGFPAEFAMYLNYCRGLRFEEAPDYMYLRQLFRILFRTLNHQYDYTFDWTMLKQKAAQQAASSSGQGQQAQTPTGF

SEQ ID NO: 18 - protein, 391 aa, Bos taurus
MSGPVPSRARVYTDVNTHRPREYWDYESHVVEWGNQDDYQLVRKLGRGKYSEVFEAINITNNEKVVVKILKPVKKKKIKREIKILENLRGGPNIITLADIVKDPVSRTPALVFEHVNNTDFKQLYQTLTDYDIRFYMYEILKALDYCHSMGIMHRDVKPHNVMIDHEHRKLRLIDWGLAEFYHPGQEYNVRVASRYFKGPELLVDYQMYDYSLDMWSLGCMLASMIFRKEPFFHGHDNYDQLVRIAKVLGTEDLYDYIDKYNIELDPRFNDILGRHSRKRWERFVHSENQHLVSPEALDFLDKLLRYDHQSRLTAREAMEHPYFYTVVKDQARMGSSSMPGGSTPVSSANMMSGISSVPTPSPLGPLAGSPVIAAANPLGMSVPAAAGAQQ

(19) United States
(12) Patent Application Publication
(10) Pub. No.: US 2026/0250701 A1
(43) Pub. Date: Aug. 27, 2026

(54) METHODS FOR PRODUCING FUNCTIONAL MILK PROTEINS IN A PLANT CELL, PRODUCTS AND USES THEREOF

(71) Applicant: NEW MOO FOODS LTD, Raanana (IL)

(72) Inventors: Yulia FRIDMAN, Haifa (IL); Hod YANOVER, Modiin (IL)

(21) Appl. No.: 18/993,427

(22) PCT Filed: Jul. 13, 2023
(86) PCT No.: PCT/IL2023/050732

§ 371 Date: Jan. 10, 2025

Related U.S. Application Data
(60) Provisional application No. 63/368,303, filed on Jul. 13, 2022.

(51) Int. Cl. C12N 15/82; C07K 14/47; C12N 9/12

(52) U.S. Cl. CPC C12N 15/8257; C07K 14/4732; C12N 9/12; C12Y 207/11001

(57) ABSTRACT

Provided herein are plant cells genetically modified to express at least one milk protein naturally expressed by a mammal a mammal and at least one protein promoting at least one post translation modification in said at least one milk protein, plants comprising said cells and compositions comprising said cells or any portion, or harvested product, or tissue, or isolate, or extract, or secretion, or extrudate thereof. Vectors or DNA constructs and methods for producing said genetically modified plant cells or any compositions thereof are also provided herein.

### TECHNOLOGICAL FIELD

The present invention relates to biosynthesis of functional milk proteins in non-mammal cells, such functional proteins and their use in industry.

### BACKGROUND ART

References considered to be relevant as background to the presently disclosed subject matter are listed below:
[1] John W. Holland, Mike J. Boland, Chapter 5—“Post-translational Modifications of Caseins”, Food Science and Technology, Milk Proteins (Second Edition), Academic Press, 2014, Pages 141-168
[2] Philip R, Darnowski D W, Maughan P J, Vodkin L O. “Processing and localization of bovine beta-casein expressed in transgenic soybean seeds under control of a soybean lectin expression cassette”. Plant Sci. 2001 July; 161 (2): 323-335.
[3] Knoop, A.-M., Knoop, E., Wiechen, A. “Sub-structure of synthetic casein micelles”. Journal of Dairy Research. 1979; 46, 347-350
[4] Akoi, T. “Incorporation of individual casein constituents into casein aggregates cross-linked by colloidal calcium phosphate in artificial casein micelles”. Journal of Dairy Research. 1989; 56, 613-618
[5] WO 2022/098853

### BACKGROUND

Bovine caseins are the major protein group in the milk, comprising 80% of total milk proteins. There are 4 main caseins in the milk, named αS1, αS2, β and κ-caseins. In the process of protein maturation, the caseins undergo various post translation modifications (PTMs) [1], that facilitate coherent folding of the caseins. This 3D structure is necessary for the formation of functional casein micelle, a quaternary structure of the caseins. The stability of a casein micelle, or its controlled destabilization in the case of cheese and yoghurt manufacture, is of primary concern to the dairy industry.

When expressing caseins in foreign host, one has to verify coherent tertiary structure. When checking soybean as a potential host, it is known that the proteins do not undergo coherent PTMs, thus resulting in an altered folding [2].

In addition to expression, there is also a need that the casein proteins be functional, i.e. form into functional micelles. The formation of artificial casein micelles has been described [3][4]. WO 2022/098853 discloses that recombinantly made kappa casein lacking PTMs can form stable micelles [5].

There is a need for suitable expression systems in order to obtain functional caseins and production of functional micelles that may be used to produce milk alternatives and various food derivatives from animal-free systems.

### SUMMARY OF THE INVENTION

The present disclosure relates to plant cells that are genetically modified to express at least one milk protein that is naturally expressed by a mammal and at least one protein promoting at least one post translation modification (PTM) in said expressed at least one milk protein.

Also provided by the present disclosure is a plant comprising at least one plant cell that is genetically modified to express at least one milk protein that is naturally expressed by a mammal and at least one protein promoting at least one post translation modification (PTM) in said expressed at least one milk protein.

Further disclosed herein is a composition comprising at least one plant cell genetically modified to express at least one milk protein that is naturally expressed by a mammal (and not in plants, under natural conditions) and at least one protein promoting at least one post translation modification (PTM) in said expressed at least one milk protein. The composition can be, for example, a cell culture.

Further disclosed herein is a composition comprising a plant portion comprising at least one artificially produced milk protein.

Also disclosed herein is a vector encoding for at least one milk protein that is naturally expressed by a mammal and at least one protein promoting at least post translation modification (PTM) of said expressed milk protein.

The plant cell disclosed herein can also be one comprising the vector disclosed herein.

Also disclosed herein is a method of producing a composition comprising at least one plant cell genetically modified to express at least one milk protein naturally expressed by a mammal and at least one protein promoting at least one post translation modification (PTM) in said expressed at least one milk protein, the method comprising:
(a) providing at least one vector for expressing in a plant cell, at least one milk protein naturally expressed by a mammal and at least one protein promoting at least post translation modification (PTM) in said milk protein;
(b) transfecting at least one plant cell with said at least one vector;
(c) providing conditions suitable for (i) expressing the at least one milk protein and the at least one protein promoting at least post translation modification (PTM) in said milk protein, and (ii) suitable for promoting said PTM in said expressed milk protein.

Yet further, disclosed herein is an artificially produced milk protein obtained or obtainable from a plant source, wherein said milk protein is coagulable, and the milk protein being one that is naturally expressed by a mammal.

Finally, disclosed herein is a plant part or portion comprising at least one artificially produced milk protein that is coagulable, wherein said plant comprises at least one cell disclosed herein.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1: Schematic representation of the three types of vectors constructed for expression of αS1-casein into soybean.

FIG. 2: Schematic representation of the three types of vectors constructed for expression of αS2-casein into soybean.

FIG. 3: Schematic representation of the three types of vectors constructed for expression of β-casein into soybean.

FIG. 4: Schematic representation of the three types of vectors constructed for expression of κ-casein into soybean.

FIG. 5A-5D: Western analysis of native-PAGE of αS1, αS2, β and κ caseins, expressed alone or in combination with either CSNK1 or CSNK2.

FIG. 5A: Picture of Western analysis of native-PAGE of αS1 casein.

FIG. 5B: Picture of Western analysis of native-PAGE of αS2 casein.

FIG. 5C: Picture of Western analysis of native-PAGE of β casein.

FIG. 5D: Picture of Western analysis of native-PAGE of κ casein.

Arrows indicate the relevant bands, since staining is not specific. As a reference (STD, standard), bovine caseins were run alongside analyzed protein sample.

FIG. 6: Picture showing the difference between light refraction of casein micelles (right) and of single and separated caseins (left).

FIG. 7A-7B: Optigraph analysis (curve and curd profile).

FIG. 7A: Picture of Optigram analysis.

FIG. 7B: Pictures of cuvettes. Different micellization protocols (cuvettes 1-6 correspond to cuvettes 1-4 on the Optigram), enzymatic curd profile of bovine micelles (cuvettes 7-9 correspond to cuvette 6 on the Optigram) and singular caseins (cuvette 10 corresponds to cuvette 5 on the Optigram).

FIG. 8A-8C: Pictures under TEM microscopy of different micelles.

FIG. 8A: Singular caseins in soy beans extract. Random small aggregates of caseins are indicated.

FIG. 8B: Casein micelles from singular caseins following micellization protocol.

FIG. 8C: Bovine casein micelles.

FIG. 9A-9B: Pictures illustrating length of stretched cheese in 250° C.

FIG. 9A: Picture of melted cheese.

FIG. 9B: Picture of stretched melted cheese.

### DETAILED DESCRIPTION

The present disclosure provides suitable expression systems in order to obtain functional caseins in plant hosts. These functional caseins may then be used for the production of functional micelles which are necessary to produce milk alternatives and various food derivatives from animal-free systems.

Thus, in a first aspect, the present disclosure relates to plant cells that are genetically modified to express at least one milk protein that is naturally expressed by a mammal and at least one protein promoting at least one post translation modification (PTM) in said expressed at least one milk protein.

In some further embodiments, the plant cells may express at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty milk proteins that are naturally expressed by a mammal.

In some embodiments, the plant cells may express at least two milk proteins that are naturally expressed by a mammal. In some embodiments, the plant cells may express at least three milk proteins that are naturally expressed by a mammal. In some embodiments, the plant cells may express at least four milk proteins that are naturally expressed by a mammal. In some embodiments, the plant cells may express at least five milk proteins that are naturally expressed by a mammal. In some embodiments, the plant cells may express at least six milk proteins that are naturally expressed by a mammal. In some embodiments, the plant cells may express at least seven milk proteins that are naturally expressed by a mammal. In some embodiments, the plant cells may express at least eight milk proteins that are naturally expressed by a mammal. In some embodiments, the plant cells may express at least nine milk proteins that are naturally expressed by a mammal. In some embodiments, the plant cells may express at least ten milk proteins that are naturally expressed by a mammal.

In some further embodiments, the plant cells may express at least two, or at least three, or at least four, or at least five, or at least six, or at least seven, or at least eight, or at least nine, or at least ten protein promoting at least one post translation modification (PTM).

In some embodiments, the plant cells may express at least two protein promoting at least one post translation modification (PTM). In some embodiments, the plant cells may express at least three protein promoting at least one post translation modification (PTM).

As used herein, the term “milk” is the normal mammary secretion of lactating female mammals, including, but not limited to, “the normal mammary secretion of milking animals” (FAO, Codex Alimentarius, “Milk” (Codex Stan 206-1999)).

“Milk proteins” include proteins found in milk. Still further, the term “milk proteins” refers to proteins or protein equivalents and variants found in milk such as casein, whey or the combination of casein and whey, including their subunits, which are derived from various sources and as further defined herein. Specifically, the term “milk protein” means a protein that is found in a mammal-produced milk or a protein having a sequence that is at least 80 percent identical (e.g., at least 85 percent, at least 90 percent, at least 95 percent, at least 96 percent, at least 97 percent, at least 98 percent, or at least 99 percent identical) to the sequence of a protein that is found in a mammal-produced milk. Examples of milk proteins include, but are not limited to αS1-casein, αS2-casein, β-casein, κ-casein, α-lactalbumin, β-lactoglobulin, lactoferrin, transferrin, and serum albumin. Additional milk proteins are known in the art.

In some embodiments, the expressed milk protein is functional.

As used herein, the term “functional” when relating to milk proteins, refers to properly folded and biologically active milk proteins, e.g. capable of forming functional micelles or coagulable. In some embodiments, the formation of functional micelles or coagulation does not occur in vivo, i.e. into the plant cell.

As used herein, the term “coagulable” when relating to milk protein refers to the ability of certain proteins in milk, e.g. casein, to form a gel-like clot or coagulate when acted upon by specific enzymes or acids. Coagulation is an essential process in cheese-making, where milk is curdled to separate the solids (curds) from the liquid (whey). This coagulation process is helps retain the milk solids, trap fat, and expel whey. The coagulated curd is then further processed and shaped to produce various types of cheese, or additional types of dairy products, with the texture and flavor influenced by factors such as coagulation time, temperature, and the presence of other enzymes or cultures.

Milk coagulation is a result of specific enzymatic reaction or pH reduction that breaks the suspension and lead to casein micelles stability lose, followed by huge chunks of micelles sedimentation. This sediment is called CURD or coagulum and is the base of every cheese-soft, semi-hard and hard cheese. The ability to create a typical curd, similar to dairy curd, is the desired functionality of casein micelles.

These functional milk proteins expressed in plant cells and/or derived functional micelles may then be used for the production of “milk substitute” also named “milk alternative” or for the production of “dairy-like product”.

The term “milk substitute” also named “milk alternative” refers to a composition that resembles, is similar to, is equivalent to, or is nearly identical to a dairy milk. A “milk substitute” or “milk alternative” may be preferred or necessary in situations, e.g., in which an individual is unable to consume milk due to lactose intolerance or an allergy, where milk/breastmilk is unavailable for an individual for whom milk/breastmilk is necessary or preferable, or as a preferred nutritional component for a human or non-human animal.

“Dairy-like products” as used herein refers to food substitutes that are designed to imitate or resemble traditional dairy products, such as milk, cheese, yogurt, and butter, but are made entirely from non-animal-based ingredients. These products are developed to cater to individuals who follow a vegan or lactose-free diet, are allergic to dairy, or choose to avoid animal products for ethical or environmental reasons.

In some embodiments, the dairy-like product may be milk and products derived from milk, including but not limited to yogurt, cheese (e.g., whey cheese such as ricotta; pasta filata cheese such as mozzarella; semi-soft cheese; hard cheese; washed curd cheese; soft ripened cheese; fresh cheese such as cottage cheese, feta cheese, cream cheese, and curd), dairy-based sauces, dairy spreads, cream, frozen confections (e.g., ice cream, smoothie, milk shake, frozen yogurt), dairy desserts (e.g., fresh, refrigerated, or frozen), butter (e.g., whipped butter, cultured butter), dairy powders, infant formula, milk protein concentrate, milk protein, whey protein concentrate, whey protein isolate, nutritional supplements, texturizing blends, flavoring blends, coloring blends, puddings, gels, chewables, crisps, and bars.

In some specific embodiment, the dairy-like product may be a cheese. In some more specific embodiment, the dairy-like product may be mozzarella.

As used herein, the term “Post-translational modification (PTM)” refers to the covalent and generally enzymatic modification of proteins following protein biosynthesis. This process occurs mainly in the endoplasmic reticulum and the Golgi apparatus.

Post-translational modifications can occur on the amino acid side chains or at the protein's C- or N-termini. Phosphorylation is a very common mechanism for regulating the activity of enzymes and is the most common post-translational modification. Many proteins also have carbohydrate molecules attached to them in a process called glycosylation, which can promote protein folding and improve stability as well as serving regulatory functions. The formation of disulfide bonds from cysteine residues may also be referred to as a post-translational modification. Attachment of lipid molecules, known as lipidation, often targets a protein or part of a protein attached to the cell membrane.

Post-translational modifications (PTMs) such as phosphorylation, glycosylation and possibly disulfide bond formation play a critical role in micelle formation and stability, specifically casein micelles. Phosphorylation of the α, β and/or κ caseins and glycosylation of κ-casein are well-known modifications and are critical for the formation and stability of casein micelles.

In some embodiments, the PTM may be any one of phosphorylation, glycosylation, and addition of disulfide bond. In some specific embodiments, the at least one milk protein expressed in said plant cell is phosphorylated. In some further embodiments, the protein promoting PTM is a kinase.

In some embodiments, the kinase is not from human. In some embodiments, the kinase is not a tyrosine kinase. In some embodiments, the kinase is not a serine/threonine kinase e.g. FAM20A or FAM20C. In some further embodiments, the kinase is not Casein Kinase II from human.

In some embodiments, the kinase is from Bos taurus.

In some further specific embodiments, the at least one milk protein expressed in said plant cell is a casein. In some particular embodiments, the protein promoting PTM is a casein kinase. In some specific embodiments, the protein promoting PTM is a casein kinase from Bos taurus.

Casein proteins refers to a family of related phosphoproteins (αS1, αS2, β and κ-caseins) that are commonly found in mammalian milk, comprising about 80% of the proteins in cow's milk and between 20% and 60% of the proteins in human milk. Sheep and buffalo milk have a higher casein content than other types of milk with human milk having a particularly low casein content. Casein proteins have a wide variety of uses, from being a major component of cheese, to use as a food additive.

As used herein, casein kinases refer to enzymes that catalyze the transfer of the terminal phosphoryl group of ATP to specific serine residues in dephosphorylated caseins. Protein functions such as binding, stabilization, biological activity, interactions with proteins and other biomolecules are regulated by phosphorylation-dephosphorylation of the casein proteins. Phosphorylation stabilizes calcium phosphate nano clusters in casein micelles.

As used herein, a micelle refers to a network of protein molecules held together by a combination of hydrophobic interactions between protein molecules and electrostatic interactions.

Specifically, casein micelles are particles of colloidal size that can be described as supramolecules, or a system consisting of multiple molecular entities held together and organized by means of noncovalent intermolecular binding interactions i.e. between phosphoserine-rich regions of the αS1—, αS2— and β-caseins and micellar calcium phosphate. Still further, the hydrophilic C-terminal portion of κ-casein extends from the surface, providing steric and electrostatic repulsion, which prevents micelle aggregation.

The predominant form of αS1-casein in bovine milk contains eight phosphate groups. A minor form of αS1-casein with nine phosphates, originally called αS0-casein, also occurs in bovine milk.

The αS2-casein component of bovine milk is more varied than the αS1-casein component. It generally presents as a mixture of four phosphoforms with 10-13 phosphates. A second PTM on αS2-casein is the formation of an intramolecular disulfide bond between the two cysteine residues in the protein which may contribute to micelle stability.

Bovine β-casein is a phosphoprotein that is modified post-translationally by the covalent coupling of five phosphate groups to serine residues at the N-terminal region of the protein. The phosphoserine residues of the bovine β-casein play an essential role in the formation of casein micelles via Ca²⁺-phosphate clusters and also contribute to increased curd tension during cheese making.

The κ-casein does not contain any phosphoserine clusters and appears to play a little part in calcium binding. Its major feature is a variable degree of glycosylation. κ-casein appears to be constitutively phosphorylated at Ser 170 and only partially phosphorylated at Ser 148. A minor tri-phosphorylated form has also been detected.

Still further, whereas about 40% of κ-casein has been estimated to be non-glycosylated, the remaining 60% has up to six glycans attached. The major glycan is a tetrasaccharide composed of galactose (Gal), N-cetylgalactosamine (GalNAc) and sialic or neuraminic acid (NeuAc) of the form NeuAca(2-3)Galβ(1-3)[NeuAca(2-6)]GalNAc, but monosaccharide (GalNAc), disaccharide (Galβ(1-3)GalNAc) and trisaccharide (NeuAca(2-3)Galβ(1-3)GalNAc or Galβ(1-3)[NeuAca(2-6)]GalNAc) are also found.

Furthermore, κ-casein purified from bovine milk occurs as both monomeric forms and oligomeric forms with up to eight or more monomers linked by disulfide bonds. There are only two cysteine residues (Cys 32 and Cys 109) in bovine κ-casein and they appear to be randomly linked in disulfide bonds in oligomeric forms.

As mentioned, the plant cells according to the present disclosure are genetically modified to express at least one milk protein that is naturally expressed by a mammal. As used herein, the term “Mammals” (class “Mammalia”) refers to endothermic vertebrates usually characterized by the presence of hair, three middle-ear bones, a neocortex, and in female mammals, mammary glands that secrete milk during lactation. With a few exceptions, mammals are viviparous. Mammals include, but are not limited to cows, humans, buffalo, goats, sheep, camels, dromedaries, donkeys, horses, reindeer, yaks, moose, bison, bison/cow hybrids, pigs, dogs, cats, lions, tigers, panda bears, leopards, giraffes, whales, and dolphins.

Most commercially produced milk in Europe and North America is from the Bovidae biological family of cloven-hoofed, ruminant mammals, which includes, but is not limited to, cattle (e.g., domestic cows, Bos taurus), buffalo (e.g., water buffalo [e.g., Bubalus bubalis] and African/Cape buffalo [e.g., Syncerus caffer]), goats (e.g., domestic goats, Capra aegagrus), sheep (e.g., domestic sheep, Ovis aries), bison (e.g., Bison genus, American bison, European bison), yak (e.g., Bos grunniens), and bison/cow hybrids. Common non-Bovidae sources of commercial milk include, but are not limited to, members of the Camelidae (camels, dromedaries), Equidae (donkeys, horses), Cervidae (reindeer), and Suidae (pigs) families. Other sources of milk protein of particular interest include, but are not limited to humans, dogs, and cats.

In some embodiments, the milk protein expressed in said plant cell is one that is naturally produced by a mammal selecting from the group consisting of cow, buffalo, goat, sheep bison, yak, camel, horse, cervid and pig.

In some embodiments, the milk protein expressed in said plant cell is one that is naturally produced by Bos taurus.

In some specific embodiments, the milk protein expressed in said plant cell is one that is naturally produced by human.

In some particular embodiments, the at least one milk protein is selected from the group consisting of αS1-casein, αS2-casein, β-casein and κ-casein.

In some specific embodiments, the αS1-casein comprises an amino acid sequence as denoted by SEQ ID NO: 9, said αS2-casein comprises an amino acid sequence as denoted by SEQ ID NO: 10, said β-casein comprises an amino acid sequence as denoted by SEQ ID NO: 11 and/or said κ-casein comprises an amino acid sequence as denoted by SEQ ID NO: 12.

In some specific embodiments, the αS1-casein consists of an amino acid sequence as denoted by SEQ ID NO: 9, said αS2-casein consists of an amino acid sequence as denoted by SEQ ID NO: 10, said β-casein consists of an amino acid sequence as denoted by SEQ ID NO: 11 and/or said κ-casein consists of an amino acid sequence as denoted by SEQ ID NO: 12.

In some further specific embodiments, the αS1-casein is encoded by a nucleic acid molecule comprising an nucleic acid sequence as denoted by SEQ ID NO: 2, said αS2-casein is encoded by a nucleic acid molecule comprising an nucleic acid sequence as denoted by SEQ ID NO: 4, said β-casein is encoded by a nucleic acid molecule comprising an nucleic acid sequence as denoted by SEQ ID NO: 6 and/or said κ-casein is encoded by a nucleic acid molecule comprising an nucleic acid sequence as denoted by SEQ ID NO: 8.

In some further embodiments, the αS1-casein is encoded by a nucleic acid molecule consisting of an nucleic acid sequence as denoted by SEQ ID NO: 2, said αS2-casein is encoded by a nucleic acid molecule consisting of an nucleic acid sequence as denoted by SEQ ID NO: 4, said β-casein is encoded by a nucleic acid molecule consisting of an nucleic acid sequence as denoted by SEQ ID NO: 6 and/or said κ-casein is encoded by a nucleic acid molecule consisting of an nucleic acid sequence as denoted by SEQ ID NO: 8.

In some specific embodiments, the casein kinase expressed in said genetically modified plant cell is from Bos taurus. In some further embodiments, the casein kinase is any one of casein kinase 1, casein kinase 2 also named casein kinase type I, or casein kinase type II. In some further specific embodiments, the casein kinase 1 comprises an amino acid sequence as denoted by SEQ ID NO: 17 and/or said casein kinase 2 comprises an amino acid sequence as denoted by SEQ ID NO: 18. In some further particular embodiments, the casein kinase 1 is encoded by a nucleic acid molecule comprising a nucleic acid sequence as denoted by SEQ ID NO: 14 and/or said casein kinase 2 is encoded by a nucleic acid molecule comprising an nucleic acid sequence as denoted by SEQ ID NO: 16.

In some further specific embodiments, the casein kinase 1 consists of an amino acid sequence as denoted by SEQ ID NO: 17 and/or said casein kinase 2 consists of an amino acid sequence as denoted by SEQ ID NO: 18. In some further particular embodiments, the casein kinase 1 is encoded by a nucleic acid molecule consisting of a nucleic acid sequence as denoted by SEQ ID NO: 14 and/or said casein kinase 2 is encoded by a nucleic acid molecule consisting of an nucleic acid sequence as denoted by SEQ ID NO: 16.

In yet another particular embodiments, said milk protein is αs1-casein and said protein promoting PTM is casein kinase 1 or casein kinase 2 and/or wherein said milk protein is β-casein said protein promoting PTM is casein kinase 1 or casein kinase 2.

In some further embodiments, said milk protein is αs2-casein and said protein promoting PTM is casein kinase 1 or casein kinase 2. In some further embodiments, said milk protein is κ-casein and said protein promoting PTM is casein kinase 1 or casein kinase 2.

In some embodiments, the genetically modified plant cell according to the preset disclosure refers to a seed, or a bean, grain, fruit, nut, legume, leaf, stem or root cell.

The present disclosure further provides a plant cell genetically modified to express at least one casein that is naturally expressed by a mammal (e.g. Bos taurus) and at least one kinase promoting phosphorylation in said at least one casein.

Another aspect of the present disclosure provides a plant cell genetically modified to express at least one casein that is naturally expressed by a mammal (e.g. Bos taurus) and at least one kinase promoting phosphorylation in said at least one casein, wherein said at least one casein is coagulable.

A yet another aspect of the present disclosure provides a plant cell genetically modified to express at least one of αS1-casein, αS2-casein, β-casein and κ-casein proteins that are naturally expressed by a mammal (e.g. Bos taurus) and at least one kinase promoting phosphorylation in said at least one of said casein.

A further aspect of the present disclosure refers to plant cell genetically modified to express at least one of αS1-casein, αS2-casein, β-casein and κ-casein proteins that are naturally expressed by a mammal (e.g. Bos taurus) and at least one kinase promoting phosphorylation in said at least one of said caseins, wherein said casein proteins are coagulable.

The present disclosure further provides a plant cell genetically modified to express at least one of αS1-casein, αS2-casein, β-casein and κ-casein proteins that are naturally expressed by a mammal (e.g. Bos taurus) and at least one kinase promoting phosphorylation in said at least one of said casein, wherein said casein proteins are capable of producing functional micelles.

In still another aspect, the present disclosure further provides a plant comprising the above described genetically modified plant cells.

In some embodiments, the plant may be referred to as a genetically modified plant or a transgenic plant.

The term “genetically modified plant” refers to a plant comprising at least one cell genetically modified by human. The genetic modification includes modification of an endogenous gene(s) or an endogenous chloroplast gene(s) (Day et al. (2011) Plant Biotechnol. J 9:540-553 [“Day 2011”]), for example by introducing mutation(s) deletions, insertions, transposable element(s) and the like into an endogenous polynucleotide or gene of interest. Additionally, or alternatively, the genetic modification includes transforming the plant cell with heterologous polynucleotide. A skilled artisan would appreciate that a comparison of a “genetically modified plant” to a “corresponding unmodified plant” as used herein encompasses comparing a plant comprising at least one genetically modified cell and to a plant of the same type lacking the modification.

One of ordinary skill in the art would appreciate that a genetically modified plant may encompass a plant comprising at least one cell genetically modified by man. In some embodiments, the genetic modification includes modification of an endogenous gene(s), for example by introducing mutation(s) deletions, insertions, transposable element(s) and the like into an endogenous polynucleotide or gene of interest. Additionally, or alternatively, in some embodiments, the genetic modification includes transforming at least one plant cell with a heterologous polynucleotide or multiple heterologous polynucleotides. The skilled artisan would appreciate that a genetically modified plant comprising transforming at least one plant cell with a heterologous polynucleotide or multiple heterologous polynucleotides may in certain embodiments be termed a “transgenic plant”.

The skilled artisan would appreciate that the term “transgenic” when used in reference to a plant as disclosed herein encompasses a plant that contains at least one heterologous transcribable polynucleotide in one or more of its cells. The term “transgenic material” encompasses broadly a plant or a part thereof, including at least one cell, multiple cells or tissues that contain at least one heterologous polynucleotide in at least one of cell. Thus, comparison of a “transgenic plant” and a “corresponding non transgenic plant”, or of a “genetically modified plant comprising at least one cell having altered expression, wherein said plant comprising at least one cell comprising a heterologous transcribable polynucleotide” and a “corresponding unmodified plant” encompasses comparison of the “transgenic plant” or “genetically modified plant” to a plant of the same type lacking said heterologous transcribable polynucleotide. A skilled artisan would appreciate that, in some embodiments, a “transcribable polynucleotide” comprises a polynucleotide that can be transcribed into an RNA molecule by an RNA polymerase.

In some embodiments the plant may be a genetically modified soybean.

Alternatively, non-soy plants (e.g., nicotine, rice, peanuts, pea) may also be used. In some embodiments, the plant is a tobacco plant. In some embodiments, the plant is a rice plant. In some embodiments, the plant is a peanut plant. In some embodiments, the plant is a pea plant.

In a further aspect, the present disclosure provides a composition comprising at least one plant cell genetically modified to express at least one milk protein that is naturally expressed by a mammal and at least one protein promoting at least post translation modification (PTM) in said expressed at least one milk protein. The composition can be, for example, a cell culture.

In some embodiments, the composition may comprise a portion of a plant comprising said plant cell.

As used herein, the portion of said plant includes but is not limited to harvested products, tissue, isolate, extract, secretion, extrudate etc.

In some embodiments, the composition according to the present disclosure comprises the above described genetically modified plant cells.

The present disclosure further provides a composition comprising a plant portion comprising at least one artificially produced milk protein. In some embodiments, the artificially produced milk protein is coagulable. In some embodiments, the composition comprising a plant portion as defined herein comprises at least one plant cell as defined above in the present aspect of the present disclosure, or any harvested product, or tissue, or isolate, or extract, or secretion, or extrudate thereof.

In some embodiments, the composition is any one of a food, a medicament or a cosmetic.

In some specific embodiments, the composition is a milk composition or a milk substitute/milk alternative or a dairy-like product.

In some embodiments, the composition is comprised into a milk composition or a milk substitute/milk alternative or a dairy-like product.

Methods for obtaining said milk composition or milk substitute/milk alternative or dairy-like product include, but are not limited to, isolation, extraction, exudation (e.g., from a plant root), or secretion, as well as ingestion, with or without grinding or filtering, of the plant, or of a seed, bean, grain, fruit, nut, legume, leaf, stem, root, portion, or product thereof.

According to the present disclosure, milk from a mammal may be further added to the food, medicament, cosmetic composition derived from the genetically modified plant cell or plant or product thereof to provide, e.g., stability, consistency, flavor, or other qualities associated with milk from a mammal. Milk from a mammal may be added to the food, medicament, cosmetic composition for a final concentration of 1 percent, 2 percent, 3 percent, 5 percent, 10 percent, 15 percent, 20 percent, 25 percent, 30 percent, 35 percent, 40 percent, 45 percent, 50 percent, 55 percent, 60 percent, 65 percent, 70 percent, 75 percent, 80 percent, 85 percent, 90 percent, 95 percent, 97 percent, 98 percent, 99 percent milk from a mammal. An unmodified milk alternative from a plant may be added to the food, medicament, cosmetic composition for a final concentration of 1 percent, 2 percent, 3 percent, 5 percent, 10 percent, 15 percent, 20 percent, 25 percent, 30 percent, 35 percent, 40 percent, 45 percent, 50 percent, 55 percent, 60 percent, 65 percent, 70 percent, 75 percent, 80 percent, 85 percent, 90 percent, 95 percent, 97 percent, 98 percent, 99 percent milk alternative from a plant cell or plant.

Methods of making a dairy-like product or dairy-like ingredient may include adding additional components or ingredients to the compositions disclosed herein. Additional components/ingredients may be added such as lipids (e.g. fats and oils), carbohydrates (e.g. sugars).

In some embodiments, lipids can be added. In some cases, lipids may be essentially free of animal-obtained fats and/or oils. lipids used herein may include plant-based lipids (vegetable lipids) such as canola oil, sunflower oil, coconut oil, palm oil, and any combinations thereof. The concentration of the lipids may be about 0% to about 5% in the composition. The concentration of lipids may be at least 0.5% or about 1%. The concentration of lipids may be at most 5%. The concentration of lipids may be about 0%, about 0, 1%, about 0. 5%, about 1%, about 2%, about 3%, about 4%, or about 5% The concentration of lipids may be from 0 to 0 5%, 0 5% to 1%, 1% to 3%, 1% to 4%, or 1% to 5%. The concentration of lipids may be at most 2%, 3%, 4%, or 5%.

The composition as described herein may further comprise one or more carbohydrates. carbohydrates used herein may include plant-based carbohydrates (e.g., plant-based monosaccharides, disaccharides, oligosaccharides and/or polysaccharides). Examples of carbohydrates include, without being limited thereto sucrose, glucose, fructose, galactose, lactose, maltose, mannose, allulose, tagatose, xylose, and arabinose. The concentration of carbohydrates may be about 0% to about 5% in the composition. The concentration of carbohydrates may be at least 0.5% or about 1%. The concentration of carbohydrates may be at most 5%. The concentration of carbohydrates may be about 0%, about 0, 1%, about 0.5%, about 1%, about 1.5%, about 2%, about 3%, about 4%, or about 5% The concentration of carbohydrates may be from 0 to 0 5%, 0 5% to 1%, 1% to 3%, 1% to 4%, or 1% to 5%. The concentration of carbohydrates may be at most 1.5%, 2%, 3%, 4%, or 5%.

In some case, fats may be emulsified into the compositions that are in the form of a liquid colloid using a sonication, sheer mixing under temperature treatment, or high-pressure homogenization process. An emulsifier such as soy lecithin or xanthan gum may be used to secure a stable emulsion.

In some embodiments, the composition may be treated to form a coagulated colloid. In certain embodiments, the treatment is a reduction of pH of the liquid colloid. The reduction of pH of the liquid colloid to generate coagulated colloid may be conducted by adding one or more acids or acidifying with one or more microorganisms.

In some embodiments, the pH is adjusted at a range of about 6 to 7. In some particular embodiments, the pH is adjusted at 6.7 or 6.3.

In some embodiments, the composition may be a micelle composition that comprises at least two caseins. In some further embodiments, the micelle composition comprises at least three caseins. In some embodiments, the micelle composition comprises four caseins, i.e. αS1-casein, αS2-casein, β-casein and κ-casein. In some specific embodiments, the ratio between αS1:αS2:β:κ casein for micelle formation is about 4:1:4:1. In some further embodiments, the ratio between αS1:αS2:B:κ casein for micelle formation is about 2:1:2:1. In some other embodiments, the ratio between αS1:αS2:B:κ casein for micelle formation is about 3:1:3:1. In some further embodiments, the ratio between αS1:αS2:B:κ casein for micelle formation may vary from 30% to 50% to the above mentioned ratios i.e. 30-50% of about 4:1:4:1 or 30-50% of about 2:1:2:1 or 30-50% of about 3:1:3:1.

In some further embodiments, the composition or micelle composition further comprises at least one salt selected from the group consisting of a calcium salt, a citrate salt, and a phosphate salt.

In some embodiments, the composition or micelle composition may be susceptible to renneting, e.g. by treatment with a renneting agent (e.g. Chymosin). In some further embodiment, the composition or micelle composition after renneting, may form stable and strong curds.

In some other embodiments, the composition is a dairy-like product, specifically a cheese.

In some embodiments, said cheese may be selected from the group consisting of a soft cheese, a hard cheese, a salted cheese, a pasta filata cheese, an aged cheese, a ripened cheese, mozzarella, paneer, cream cheese, cottage cheese, an aged or matured cheese selected from the group consisting of cheddar, Swiss, gouda, brie, camembert, feta, halloumi, edam, manchego, colby, muenster, blue cheese, or parmesan. In some specific embodiments, the cheese is mozzarella.

In some further embodiments, the cheese is capable of one or more of stretching when heated, melting when heated, or browning when heated.

Conventional methods to test the above properties i.e. stretching, melting or browning of the cheese are disclosed herein in Example 9 and Table 6.

As used herein, the term “stretching” refers to the texture and ability of certain cheeses to stretch when heated. When subjected to heat, these cheeses soften, become pliable, and form long, stretchy strands or strings. This characteristic is desirable in certain types of cheese, particularly those used in dishes like pizza, lasagna, or grilled cheese sandwiches, as the stretching enhances the cheese's mouthfeel and adds a visually appealing aspect to the food.

In the food industry, the term “stretching” is further commonly associated with the following general terms: meltability, stringiness, elasticity, taffy-like texture. “Meltability” refers to the ability of a cheese to melt smoothly when exposed to heat, forming a creamy or gooey texture. Cheeses with good meltability are often used for toppings, fillings, or sauces. “Stringiness” describes the formation of long, stringy strands when the cheese is heated and pulled apart. Stringiness is desirable in cheeses like mozzarella or provolone, which are known for their ability to stretch. “Elasticity” refers to the cheese's ability to return to its original shape after being stretched. Cheeses with high elasticity will bounce back and maintain their form, contributing to the desirable texture and mouthfeel in dishes. “Taffy-like texture” is used to describe the stretchy, chewy, and malleable texture of certain cheeses when melted or heated. It implies a pliable consistency that can be pulled or manipulated.

In some embodiments, the meltability and/or stringiness and/or elasticity and/or taffy-like texture of the cheese is comparable to an animal-obtained dairy cheese.

In some further embodiments, the meltability and/or stringiness and/or elasticity and/or taffy-like texture of the cheese is improved compared to an animal-obtained dairy cheese.

The present disclosure further provides a composition comprising a plant portion comprising at least one casein that is naturally produced by a mammal (e.g. Bos taurus), wherein said at least one casein is coagulable.

Another aspect of the present disclosure relates to a composition comprising a plant or a portion, or a harvested product, or a tissue, or an isolate, or an extract, or a secretion, or an extrudate thereof, comprising at least one casein that is naturally produced by a mammal (e.g. Bos taurus), wherein said at least one casein is coagulable.

Another aspect of the invention relates to a composition comprising a plant or a portion, or a harvested product, or a tissue, or an isolate, or an extract, or a secretion, or an extrudate thereof, comprising at least one of αS1-casein, αS2-casein, β-casein and κ-casein proteins that are naturally produced by a mammal (e.g. Bos taurus), wherein said casein proteins are coagulable.

A yet additional aspect of the invention provides a dairy-like product comprising at least one plant cell genetically modified to express at least one casein that is naturally expressed by a mammal (e.g. Bos taurus) and at least one kinase promoting phosphorylation in said at least casein.

The present disclosure also refers to a dairy-like product comprising at least one plant cell genetically modified to express at least one casein that is naturally expressed by a mammal (e.g. Bos taurus) and at least one kinase promoting phosphorylation in said at least casein, wherein said at least one casein is coagulable.

Another aspect of the present disclosure relates to a dairy-like product comprising at least one plant cell genetically modified to express at least one of αS1-casein, αS2-casein, β-casein and κ-casein proteins that are naturally expressed by a mammal (e.g. Bos taurus) and at least one kinase promoting phosphorylation in said at least one of said caseins, wherein said casein proteins are coagulable.

A yet another aspect of the present disclosure relates to a dairy-like product comprising at least one plant cell genetically modified to express at least one of αS1-casein, αS2-casein, β-casein and κ-casein proteins that are naturally expressed by a mammal (e.g. Bos taurus) and at least one kinase promoting phosphorylation in said at least one of said caseins, wherein said casein proteins are capable of producing functional micelles.

A further aspect of the present disclosure provides a dairy-like product comprising a plant portion comprising at least one casein that is naturally produced by a mammal (e.g. Bos taurus), wherein said at least one casein is coagulable.

A yet further aspect of the present disclosure relates to a dairy-like product comprising a plant or a portion, or a harvested product, or a tissue, or an isolate, or an extract, or a secretion, or an extrudate thereof, comprising at least one casein that is naturally produced by a mammal (e.g. Bos taurus), wherein said at least one casein is coagulable.

The present disclosure also provides a dairy-like product comprising a plant or a portion, or a harvested product, or a tissue, or an isolate, or an extract, or a secretion, or an extrudate thereof, comprising at least one of αS1-casein, αS2-casein, β-casein and κ-casein proteins that are naturally produced by a mammal (e.g. Bos taurus), wherein said casein proteins are coagulable.

In another aspect, the present disclosure also provides a vector encoding for at least one milk protein that is naturally expressed by a mammal and at least one protein promoting at least post translation modification (PTM) of said expressed milk protein.

In some embodiments, said vector may encode for at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty milk proteins that are naturally expressed by a mammal.

In some embodiments, the vector may encode for at least two milk proteins that are naturally expressed by a mammal. In some embodiments, the vector may encode for at least three milk proteins that are naturally expressed by a mammal. In some embodiments, the vector may encode for at least four milk proteins that are naturally expressed by a mammal. In some embodiments, the vector may encode for at least five milk proteins that are naturally expressed by a mammal. In some embodiments, the vector may encode for at least six milk proteins that are naturally expressed by a mammal. In some embodiments, the vector may encode for at least seven milk proteins that are naturally expressed by a mammal. In some embodiments, the vector may encode for at least eight milk proteins that are naturally expressed by a mammal. In some embodiments, the vector may encode for at least nine milk proteins that are naturally expressed by a mammal. In some embodiments, the vector may encode for at least ten milk proteins that are naturally expressed by a mammal.

In some embodiments, said vector may encode for at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty caseins.

In some further embodiments, said vector may encode for at least two, or at least three, or at least four, or at least five, or at least six, or at least seven, or at least eight, or at least nine, or at least ten proteins promoting at least one post translation modification (PTM).

In some further embodiments, said vector may encode for at least two, or at least three, or at least four, or at least five, or at least six, or at least seven, or at least eight, or at least nine, or at least ten kinases.

In some embodiments, said vector may encode for at least two kinases. In some embodiments, said vector may encode for at least three kinases.

In some specific embodiments, said vector may encode for one milk proteins and for one protein promoting at least one post translation modification. In some more specific embodiments, said vector may encode at least one casein and at least one kinase, e.g. a casein kinase, specifically one casein and one kinase.

In some embodiments, the nucleic acid sequence encoding for the at least one milk protein naturally expressed by a mammal and the at least one protein promoting at least one post translation modification comprised in said vector may be separated by a genetic element enabling the production of multiple proteins from a single DNA transcript/construct.

In some embodiments, said genetic element enabling the production of multiple proteins from a single DNA transcript/construct may be 2A sequence. In some other embodiment, said genetic element may be an IRES sequence.

As used herein, a “2A sequence” relates to a short peptide sequence derived from the foot-and-mouth disease virus (FMDV) which enables to achieve a process called “ribosome skipping” or “self-cleavage.” The 2A sequence allows the translation of a single mRNA into multiple proteins. The mechanism behind the 2A sequence involves the ribosome encountering the 2A peptide during translation. The presence of the 2A sequence causes the ribosome to temporarily pause, resulting in the formation of a peptide bond between the carboxyl terminus of the upstream protein and the amino terminus of the downstream protein. This leads to the release of the upstream protein from the ribosome, allowing independent translation of the downstream protein.

As used herein, an “IRES sequence” or “Internal Ribosome Entry Site sequence” relates to a RNA element found in the 5′ untranslated region (UTR) of some viral and cellular mRNAs, enabling the ribosome to bypass the traditional 5′ cap-dependent translation initiation mechanism. The IRES sequence acts as a binding site for ribosomal subunits and initiation factors, allowing them to assemble and initiate translation at internal positions within the mRNA. This enables the simultaneous production of different proteins in specific cellular conditions.

In some embodiments, said vector may comprise a nucleic acid sequence encoding for αS1-casein, a nucleic acid sequence comprising a 2A sequence and a nucleic acid sequence encoding for a casein kinase 1. In some other embodiments, said vector may comprise a nucleic acid sequence encoding for αS1-casein, a nucleic acid sequence comprising a 2A sequence and a nucleic acid sequence encoding for a casein kinase 2.

In some embodiments, said vector may comprise a nucleic acid sequence encoding for αS2-casein, a nucleic acid sequence comprising a 2A sequence and a nucleic acid sequence encoding for a casein kinase 1. In some other embodiments, said vector may comprise a nucleic acid sequence encoding for αS2-casein, a nucleic acid sequence comprising a 2A sequence and a nucleic acid sequence encoding for a casein kinase 2.

In some embodiments, said vector may comprise a nucleic acid sequence encoding for β-casein, a nucleic acid sequence comprising a 2A sequence and a nucleic acid sequence encoding for a casein kinase 1. In some other embodiments, said vector may comprise a nucleic acid sequence encoding for β-casein, a nucleic acid sequence comprising a 2A sequence and a nucleic acid sequence encoding for a casein kinase 2.

In some embodiments, said vector may comprise a nucleic acid sequence encoding for κ-casein, a nucleic acid sequence comprising a 2A sequence and a nucleic acid sequence encoding for a casein kinase 1. In some other embodiments, said vector may comprise a nucleic acid sequence encoding for κ-casein, a nucleic acid sequence comprising a 2A sequence and a nucleic acid sequence encoding for a casein kinase 2.

In some embodiments, said vector is for expression in a plant cell.

In some particular embodiments, the vector is a DNA binary vector or a viral vector.

Vectors, as used herein, are nucleic acid molecules to be introduced into a host cell, thereby producing a transformed host cell. A vector may include nucleic acid sequences that permit it to replicate in a host cell, such as an origin of replication. A vector may also include one or more selectable marker genes and other genetic elements known in the art, including promoter elements that direct nucleic acid expression.

Viral vectors are useful for transformation of more transformation-resistant plants (e.g., soybean or common bean). In some embodiments, viral vectors, such as bean pod mottle virus (BPMV; genus Comovirus) vectors, are used for foreign gene expression and virus-induced gene silencing (VIGS) (Zhang et al. (May 2010) Plant Physiol. 153:52-65)). Cells are transformed, e.g., via biolistics or via direct DNA-rubbing inoculation (Zhang 2010).

In some embodiment, a gene gun or a biolistic particle delivery system (biolistics) is used for plant transformation to deliver exogenous DNA (transgenes) to cells (Rech et al. (2008) Nature Protocols 3 (3): 410-418). In some embodiments, the plasmid is designed and apical meristems of plants (e.g., soybean, bean, cotton) are bombarded with microparticle-coated DNA, followed by in vitro culture and selection of transgenic plants (Rech 2008). In other embodiments, a callus of undifferentiated plant cells or a group of immature embryos growing on gel medium in vitro. In some embodiments, the cells are then treated with a series of plant hormones, such as auxins or gibberellins to obtain plants.

In some embodiments, agroinfiltration is used to induce transient expression of genes in a plant or an isolated leaf or another portion of a plant. A suspension of Agrobacterium (e.g., Agrobacterium tumefaciens) is introduced into the plant by, e.g., direct injection or vacuum filtration, or is brought into association with plant cells immobilized on a porous support (plant cell packs). The bacteria transfer the desired gene into the plant cells via transfer of Ti plasmid-derived T-DNA.

Several methods for transforming a plant according to the teachings of the present disclosure are known to those skilled in the art. As used herein the term “transformation” or “transforming” describes a process by which a foreign DNA, such as a DNA construct, including expression vector, enters and changes a recipient cell into a transformed, genetically altered or transgenic cell.

Transformation of a cell may be stable or transient. The term “transient transformation” or “transiently transformed” refers to the introduction of one or more exogenous polynucleotides into a cell in the absence of integration of the exogenous polynucleotide into the host cell's genome. In contrast, the term “stable transformation” or “stably transformed” refers to the introduction and integration of one or more exogenous polynucleotides into the genome of a cell. The term “stable transformant” refers to a cell which has stably integrated one or more exogenous polynucleotides into the genomic or organellar DNA. It is to be understood that an organism or its cell transformed with the nucleic acids, constructs and/or vectors of the present invention can be transiently as well as stably transformed.

In some embodiments, transformation techniques including breeding through transgene editing, use of transgenes, use of transient expression of a gene or genes, or use of molecular markers, or any combination thereof, may be used in the breeding of a plant having an altered expression. If transformation techniques require use of tissue culture, transformed cells may be regenerated into plants in accordance with techniques well known to those of skill in the art. Additionally, grafting may be used to facilitate expression of proteins in trees, including nuts in nut trees. The regenerated plants may then be grown and crossed with the same or different plant varieties using traditional breeding techniques to produce seeds, beans, grains, fruits, vegetables, nuts, or legumes, which are then selected under the appropriate conditions.

In some more general aspect, the present disclosure provides a DNA construct comprising a nucleic acid sequence encoding for at least one milk protein that is naturally expressed by a mammal and at least one protein promoting at least post translation modification (PTM) of said expressed milk protein.

In some embodiments, said DNA construct may comprise a nucleic acid sequence encoding for at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty milk proteins that are naturally expressed by a mammal.

In some embodiments, said DNA construct may comprise a nucleic acid sequence encoding for at least two milk proteins that are naturally expressed by a mammal. In some embodiments, said DNA construct may comprise a nucleic acid sequence encoding for at least three milk proteins that are naturally expressed by a mammal. In some embodiments, said DNA construct may comprise a nucleic acid sequence encoding for at least four milk proteins that are naturally expressed by a mammal. In some embodiments, said DNA construct may comprise a nucleic acid sequence encoding for at least five milk proteins that are naturally expressed by a mammal. In some embodiments, said DNA construct may comprise a nucleic acid sequence encoding for at least six milk proteins that are naturally expressed by a mammal. In some embodiments, said DNA construct may comprise a nucleic acid sequence encoding for at least seven milk proteins that are naturally expressed by a mammal. In some embodiments, said DNA construct may comprise a nucleic acid sequence encoding for at least eight milk proteins that are naturally expressed by a mammal. In some embodiments, said DNA construct may comprise a nucleic acid sequence encoding for at least nine milk proteins that are naturally expressed by a mammal. In some embodiments, said DNA construct may comprise a nucleic acid sequence encoding for at least ten milk proteins that are naturally expressed by a mammal.

In some embodiments, said DNA construct may comprise a nucleic acid sequence encoding for at least two, or at least three, or at least four, or at least five, or at least six, or at least seven, or at least eight, or at least nine, or at least ten, at least eleven, or at least twelve, or at least thirteen, or at least fourteen, or at least fifteen, or at least sixteen, or at least seventeen, or at least eighteen, or at least nineteen, or at least twenty caseins.

In some further embodiments, said DNA construct may comprise a nucleic acid sequence encoding for at least two, or at least three, or at least four, or at least five, or at least six, or at least seven, or at least eight, or at least nine, or at least ten protein promoting at least one post translation modification (PTM).

In some further embodiments, said DNA construct may comprise a nucleic acid sequence encoding for at least two, or at least three, or at least four, or at least five, or at least six, or at least seven, or at least eight, or at least nine, or at least ten kinases.

In some further embodiments, said DNA construct may comprise a nucleic acid sequence encoding for one milk protein and for one protein promoting at least one post translation modification. In some further embodiments, said DNA construct may comprise a nucleic acid sequence encoding for at least one casein and at least one kinase, e.g. a casein kinase, specifically one casein and one kinase.

In some embodiments, the nucleic acid sequence encoding for the at least one milk protein naturally expressed by a mammal and the at least one protein promoting at least one post translation modification comprised in said DNA construct may be separated by a genetic element enabling the production of multiple proteins from a single DNA transcript/construct.

In some embodiments, said genetic element enabling the production of multiple proteins from a single DNA transcript/construct may be 2A sequence. In some other embodiment, said genetic element may be an IRES sequence.

In some embodiments, said DNA construct may comprise a nucleic acid sequence encoding for αS1-casein, a nucleic acid sequence comprising a 2A sequence and a nucleic acid sequence encoding for a casein kinase 1. In some other embodiments, said vector may comprise a nucleic acid sequence encoding for αS1-casein, a nucleic acid sequence comprising a 2A sequence and a nucleic acid sequence encoding for a casein kinase 2.

In some embodiments, said DNA construct may comprise a nucleic acid sequence encoding for αS2-casein, a nucleic acid sequence comprising a 2A sequence and a nucleic acid sequence encoding for a casein kinase 1. In some other embodiments, said vector may comprise a nucleic acid sequence encoding for αS2-casein, a nucleic acid sequence comprising a 2A sequence and a nucleic acid sequence encoding for a casein kinase 2.

In some embodiments, said DNA construct may comprise a nucleic acid sequence encoding for β-casein, a nucleic acid sequence comprising a 2A sequence and a nucleic acid sequence encoding for a casein kinase 1. In some other embodiments, said vector may comprise a nucleic acid sequence encoding for β-casein, a nucleic acid sequence comprising a 2A sequence and a nucleic acid sequence encoding for a casein kinase 2.

In some embodiments, said DNA construct may comprise a nucleic acid sequence encoding for κ-casein, a nucleic acid sequence comprising a 2A sequence and a nucleic acid sequence encoding for a casein kinase 1. In some other embodiments, said vector may comprise a nucleic acid sequence encoding for κ-casein, a nucleic acid sequence comprising a 2A sequence and a nucleic acid sequence encoding for a casein kinase 2.

The term “construct” as used herein refers to an artificially assembled or isolated nucleic acid molecule which includes at least one polynucleotide of interest. In general, a construct may include the polynucleotide or polynucleotides of interest, a marker gene which in some cases can also be a gene of interest and appropriate regulatory sequences. It should be appreciated that the inclusion of regulatory sequences in a construct is optional, for example, such sequences may not be required in situations where the regulatory sequences of a host cell are to be used. The term construct includes vectors but should not be seen as being limited thereto.

As generally known in the art, in the above-described vector or construct, the different nucleic acid elements are operably linked in order to obtain efficient expression of the at least one proteins or polypeptides of interest. The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation.

The term “expression”, as used herein, refers to the production of a functional end-product e.g., an mRNA or a protein.

In some embodiments, the vector or DNA construct comprises at least one promoter. The terms “promoter element”, “promoter” or “promoter sequence” as used herein, refer to a DNA sequence that is located at the 5′ end (i.e. precedes) the coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA. Examples of promoters include, but are not limited to: the cauliflower mosaic virus Pol-III promoter CaMV-35S-promoter (p35S), Solanum lycopersicum ubiquitin promoter 10 (SIPrUbiqlO) or soybean seed-specific promoters.

In some further embodiments, the vector or DNA construct comprises at least one enhancer. As used herein, the term an “enhancer” refers to a DNA sequence which can stimulate promoter activity and may be an innate element of the promoter or a heterologous element inserted to enhance the level or tissue-specificity of a promoter.

The terms “polynucleotide”, “polynucleotide sequence”, “nucleic acid sequence”, and “isolated polynucleotide” are used interchangeably herein. These terms encompass nucleotide sequences and the like. A polynucleotide may be a polymer of RNA or DNA or hybrid thereof, that is single- or double-stranded, linear or branched, and that optionally contains synthetic, non natural or altered nucleotide bases. The terms also encompass RNA/DNA hybrids.

The above described embodiments relative to the other aspects of the invention i.e. the plant cell of the invention, plant or composition are also suitable for the following described aspects of the invention i.e. vector or DNA construct as well as methods of producing a composition.

In some further aspect, the present disclosure provides a plant cell comprising at least one vector or DNA construct comprising a nucleic acid sequence encoding for at least one milk protein that is naturally expressed by a mammal and at least one protein promoting at least post translation modification (PTM) of said expressed milk protein.

In some embodiments, the plant cell comprising the above-described vector or DNA construct.

In some further embodiments, the plant cell may comprise one, or two, or three, or four, or five, or six, or seven, or eight, or nine, or ten different types of vectors or DNA constructs. For example, in some specific embodiments, the plant cell may comprise a vector or a DNA construct encoding for one milk protein naturally expressed by a mammal and one protein promoting at least post translation modification (PTM) of said expressed milk protein and at least one additional vector expressing two or three or four or five or six or seven or eight or nine or ten additional milk proteins naturally expressed by a mammal.

In some further aspects, the present disclosure provides a plant cell comprising at least one first vector or DNA construct comprising a nucleic acid sequence encoding for at least one milk protein that is naturally expressed by a mammal and at least one second vector or DNA construct comprising a nucleic acid sequence encoding for at least one protein promoting at least post translation modification (PTM) of said expressed milk protein.

In some embodiments, said first vector or DNA construct may comprise a nucleic acid sequence encoding for at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty milk proteins that are naturally expressed by a mammal.

In some embodiments, said first vector or DNA construct may comprise a nucleic acid sequence encoding for at least two milk proteins that are naturally expressed by a mammal. In some embodiments, said first vector or DNA construct may comprise a nucleic acid sequence encoding for at least three milk proteins that are naturally expressed by a mammal. In some embodiments, said first vector or DNA construct may comprise a nucleic acid sequence encoding for at least two milk proteins that are naturally expressed by a mammal. In some embodiments, said first vector or DNA construct may comprise a nucleic acid sequence encoding for at least four milk proteins that are naturally expressed by a mammal. In some embodiments, said first vector or DNA construct may comprise a nucleic acid sequence encoding for at least two milk proteins that are naturally expressed by a mammal. In some embodiments, said first vector or DNA construct may comprise a nucleic acid sequence encoding for at least five milk proteins that are naturally expressed by a mammal. In some embodiments, said first vector or DNA construct may comprise a nucleic acid sequence encoding for at least two milk proteins that are naturally expressed by a mammal. In some embodiments, said first vector or DNA construct may comprise a nucleic acid sequence encoding for at least six milk proteins that are naturally expressed by a mammal. In some embodiments, said first vector or DNA construct may comprise a nucleic acid sequence encoding for at least two milk proteins that are naturally expressed by a mammal. In some embodiments, said first vector or DNA construct may comprise a nucleic acid sequence encoding for at least seven milk proteins that are naturally expressed by a mammal. In some embodiments, said first vector or DNA construct may comprise a nucleic acid sequence encoding for at least two milk proteins that are naturally expressed by a mammal. In some embodiments, said first vector or DNA construct may comprise a nucleic acid sequence encoding for at least eight milk proteins that are naturally expressed by a mammal. In some embodiments, said first vector or DNA construct may comprise a nucleic acid sequence encoding for at least two milk proteins that are naturally expressed by a mammal. In some embodiments, said first vector or DNA construct may comprise a nucleic acid sequence encoding for at least nine milk proteins that are naturally expressed by a mammal. In some embodiments, said first vector or DNA construct may comprise a nucleic acid sequence encoding for at least two milk proteins that are naturally expressed by a mammal. In some embodiments, said first vector or DNA construct may comprise a nucleic acid sequence encoding for at least ten milk proteins that are naturally expressed by a mammal.

In some embodiments, said first vector or DNA construct may comprise a nucleic acid sequence encoding for at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty caseins.

In some further embodiments, said second vector or DNA construct may comprise a nucleic acid sequence encoding for at least two, or at least three, or at least four, or at least five, or at least six, or at least seven, or at least eight, or at least nine, or at least ten proteins promoting at least one post translation modification (PTM).

In some further embodiments, said second vector or DNA construct may comprise a nucleic acid sequence encoding for at least two, or at least three, or at least four, or at least five, or at least six, or at least seven, or at least eight, or at least nine, or at least ten kinases.

In some further embodiments, said second vector or DNA construct may comprise a nucleic acid sequence encoding for at least two kinases. In some further embodiments, said second vector or DNA construct may comprise a nucleic acid sequence encoding for at least three kinases.

In some further embodiments, said first vector or DNA construct may comprise a nucleic acid sequence encoding for one milk protein and for one protein promoting at least one post translation modification. In some further embodiments, said second vector or DNA construct may comprise a nucleic acid sequence encoding for at least one casein and at least one kinase, e.g. a casein kinase, specifically one casein and one kinase.

In yet another aspect, the present disclosure provides a plant comprising the above described at least one cell.

Also disclosed herein is a method of producing a composition comprising at least one plant cell genetically modified to express at least one milk protein naturally expressed by a mammal and at least one protein promoting at least post translation modification (PTM) in said expressed at least one milk protein, the method comprising:
(a) providing at least one vector for expressing in a plant cell, at least one milk protein naturally expressed by a mammal and at least one protein promoting at least post translation modification (PTM) in said milk protein;
(b) transfecting at least one plant cell with said at least one vector;
(c) providing conditions suitable for (i) expressing the at least one milk protein and the at least one protein promoting at least post translation modification (PTM) in said milk protein, and (ii) suitable for promoting said PTM in said expressed milk protein.

In some further embodiments, the step (a) may comprise providing two or three, or four, or five, or six, or seven, or eight, or nine, or ten different types of vectors or DNA constructs in a plant cell, as defined above.

The present disclosure also relates to the above method for producing a composition comprising a genetically modified plant comprising said at least one cell or a composition comprising a portion, product, isolate, exudate, secretion, or extract thereof.

In some embodiments, the expressed at least one milk protein of the methods of the present disclosure is functional. In some embodiments, the expressed at least one milk protein of the methods of the present disclosure is coagulable.

In some embodiments, the vector suitable for the method according to the present disclosure is as defined above.

In some specific embodiments, the plant suitable for the methods according to the present disclosure is soybean.

In some embodiments, the composition produced in accordance with the methods of the invention is as defined above.

In some embodiments, the composition produced in accordance with the methods of the invention is coagulated.

In some embodiments, the method of the invention may further comprise the addition of lipids and/or carbohydrates, as further detailed above.

In some embodiments, the method of the invention may further comprise treatment with a renneting agent (e.g. Chymosin). In some embodiment, said treatment may be at 30-35° C.

In some embodiments, the method of the invention may further comprise treatment to form a colloid. In certain embodiments, said treatment may be a reduction of pH of the liquid colloid. In some embodiments, the reduction of pH of the liquid colloid to generate coagulated colloid may be conducted by adding one or more acids or acidifying with one or more microorganisms.

In some embodiments, the method of the invention may further comprise adjusting the pH of the composition. In some embodiments, the pH may be adjusted at a range of about 6 to 7. In some particular embodiments, the pH is adjusted at 6.7 or 6.3.

In some embodiments, the method of the invention may further comprise the addition of at least one salt selected from the group consisting of a calcium salt, a citrate salt, and a phosphate salt.

The present disclosure provides also a method of producing a composition comprising at least one plant cell genetically modified to express at least one casein naturally expressed by a mammal (e.g. Bos taurus) and at least one kinase promoting phosphorylation in said expressed at least one casein, the method comprising:
a) providing at least one vector for expressing in a plant cell, at least one casein naturally expressed by a mammal and at least one kinase promoting phosphorylation (PTM) in said kinase;
b) transfecting at least one plant cell with said at least one vector;
c) providing conditions suitable for (i) expressing the at least one casein and the at least one kinase, and (ii) suitable for promoting phosphorylation in said expressed casein.

Another aspect of the present disclosure relates to a method of producing a composition comprising at least one plant cell genetically modified to express at least one casein naturally expressed by a mammal (e.g. Bos taurus) and at least one kinase promoting phosphorylation in said expressed at least one casein, the method comprising:
a) providing at least one vector for expressing in a plant cell, at least one casein naturally expressed by a mammal and at least one kinase promoting phosphorylation (PTM) in said kinase;
b) transfecting at least one plant cell with said at least one vector;
c) providing conditions suitable for (i) expressing the at least one casein and the at least one kinase, and (ii) suitable for promoting phosphorylation in said expressed casein,
wherein said at least one casein is coagulable.

The present disclosure further provides a method of producing a composition comprising at least one plant cell genetically modified to express at least one of αs1-casein, αs2-casein, β-casein and κ-casein proteins naturally expressed by a mammal (e.g. Bos taurus) and at least one kinase promoting phosphorylation in said at least one of said casein proteins, the method comprising:
a) providing at least one vector for expressing in a plant cell, at least one casein naturally expressed by a mammal and at least one kinase promoting phosphorylation (PTM) in said kinase;
b) transfecting at least one plant cell with said at least one vector;
c) providing conditions suitable for (i) expressing the at least one casein and the at least one kinase, and (ii) suitable for promoting phosphorylation in said expressed casein, wherein said at least one of said casein proteins is coagulable.

Another aspect of the present disclosure refers to a method of producing a composition comprising at least one plant cell genetically modified to express at least one of αs1-casein, αs2-casein, β-casein and κ-casein proteins naturally expressed by a mammal (e.g. Bos taurus) and at least one kinase promoting phosphorylation in said at least one of said casein proteins, the method comprising:
a) providing at least one vector for expressing in a plant cell, at least one casein naturally expressed by a mammal and at least one kinase promoting phosphorylation (PTM) in said kinase;
b) transfecting at least one plant cell with said at least one vector;
c) providing conditions suitable for (i) expressing the at least one casein and the at least one kinase, and (ii) suitable for promoting phosphorylation in said expressed casein, wherein said at least one of said casein proteins is capable of producing functional micelles.

Yet further, disclosed herein is an artificially produced milk protein obtained or obtainable from a plant source, wherein said milk protein is coagulable, and the milk protein being one that is naturally expressed by a mammal. In some particular embodiments, said coagulation of said milk protein does not occur in vivo, i.e. into the plant cell.

As used herein, the term “artificially produced protein” refers to a protein that was produced in a host i.e. not in its natural environment or organism following artificial manipulation(s) by human. In some embodiments, an artificially produced protein has an amino acid sequence that is engineered in that it is designed and/or produced through action of the hand of man. The term “artificially produced protein” may thus also refer to a genetically engineered protein or a recombinantly produced protein.

In some embodiments, said artificially produced milk protein is a casein.

In some further embodiments, said casein is selected from the group consisting of αS1-casein, αS2-casein, β-casein and κ-casein.

In some specific embodiments, said αS1-casein comprises an amino acid sequence as denoted by SEQ ID NO: 9, said αS2-casein comprises an amino acid sequence as denoted by SEQ ID NO: 10, said β-casein comprises an amino acid sequence as denoted by SEQ ID NO: 11 and said κ-casein comprises an amino acid sequence as denoted by SEQ ID NO: 12.

In some specific embodiments, said αS1-casein consists of an amino acid sequence as denoted by SEQ ID NO: 9, said αS2-casein consists of an amino acid sequence as denoted by SEQ ID NO: 10, said β-casein consists of an amino acid sequence as denoted by SEQ ID NO: 11 and said κ-casein consists of an amino acid sequence as denoted by SEQ ID NO: 12.

In yet some further specific embodiments, said αS1-casein is encoded by a nucleic acid molecule comprising an nucleic acid sequence as denoted by SEQ ID NO: 2, said αS2-casein is encoded by a nucleic acid molecule comprising an nucleic acid sequence as denoted by SEQ ID NO: 4, said β-casein is encoded by a nucleic acid molecule comprising an nucleic acid sequence as denoted by SEQ ID NO: 6 and/or said κ-casein is encoded by a nucleic acid molecule comprising an nucleic acid sequence as denoted by SEQ ID NO: 8. In some embodiments, said αS1-casein is encoded by a nucleic acid molecule consisting of an nucleic acid sequence as denoted by SEQ ID NO: 2, said αS2-casein is encoded by a nucleic acid molecule consisting of an nucleic acid sequence as denoted by SEQ ID NO: 4, said β-casein is encoded by a nucleic acid molecule consisting of an nucleic acid sequence as denoted by SEQ ID NO: 6 and/or said κ-casein is encoded by a nucleic acid molecule consisting of an nucleic acid sequence as denoted by SEQ ID NO: 8.

In some embodiments, said protein was extracted/purified from at least one cells as defined or from a plant as defined above.

The term “protein” or “polypeptide” as used herein refers to amino acid residues, connected by peptide bonds. A protein or polypeptide sequence is generally reported from the N-terminal end containing free amino group to the C-terminal end containing free carboxyl group and may include any polymeric chain of amino acids. More specifically, “Amino acid sequence” or “polypeptide sequence” is the order in which amino acid residues connected by peptide bonds, lie in the chain in peptides and proteins. The sequence is generally reported from the N-terminal end containing free amino group to the C-terminal end containing amide.

It should be appreciated that the present disclosure encompasses any variant or derivative of the proteins or polypeptides of the invention and any polypeptides that are substantially identical or homologue to the polypeptides encoded by the nucleic acid sequence disclosed in the present disclosure. The term “variant” or “derivative” is used to define amino acid sequences (polypeptide), with any insertions, deletions, substitutions and modifications to the amino acid sequences (protein or polypeptide) that do not alter the activity of the original polypeptides. By the term “derivative” it is also referred to homologues, variants and analogues thereof. Proteins orthologs or homologues having a sequence homology or identity to the proteins of interest in accordance with the invention, may share at least 50%, at least 60% and specifically 65%, 70%, 75%, 80%, 85%, 90%, 91%, 92%, 93%, 94%, 95%, 96%, 97%, 98%, 99%, or higher, specifically as compared to the entire sequence of the proteins of interest in accordance with the invention. Specifically, homologs that comprise or consists of an amino acid sequence that is identical in at least 50%, at least 60% and specifically 65%, 70%, 75%, 80%, 85%, 90%, 91%, 92%, 93%, 94%, 95%, 96%, 97%, 98%, 99%, or higher to SEQ ID NOs 9-12 or 17-18.

In some embodiments, derivatives refer to proteins or polypeptides, which differ from the proteins specifically defined in the present invention by insertions, deletions or substitutions of amino acid residues. It should be appreciated that by the terms “insertion/s”, “deletion/s” or “substitution/s”, as used herein it is meant any addition, deletion or replacement, respectively, of amino acid residues to the proteins disclosed by the invention, of between 1 to 50 amino acid residues, between 20 to 1 amino acid residues, and specifically, between 1 to 10 amino acid residues. More particularly, insertion/s, deletion/s or substitution/s may be of any one of 1, 2, 3, 4, 5, 6, 7, 8, 9, or 10 amino acids. It should be noted that the insertion/s, deletion/s or substitution/s encompassed by the invention may occur in any position of the modified peptide, as well as in any of the N′ or C′ termini thereof.

In particular embodiments, the present disclosure relates to a functional derivative or functional fragment of the proteins specifically defined in the present invention, wherein said functional derivative or functional fragment thereof comprises an amino acid sequence that is at least about 70%, 75%, 80%, 85%, 90%, or 95%, in particular 99% identical to the amino acid sequence of the unmodified proteins and retains a biological activity qualitatively similar to that of the unmodified proteins.

In some embodiments, said functional derivative or functional fragment of the proteins defined in the present disclosure is capable of exhibiting the same biological activity as the unmodified protein.

With respect to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention.

Finally, disclosed herein is a plant part comprising at least one artificially produced milk protein that is coagulable, wherein said plant comprises at least one cell disclosed herein.

It should be noted that the definitions and embodiments stated for one aspect of the invention are applicable and relevant to all other aspects of the invention presented herein.

As used herein, the term “or” means one or a combination of two or more of the listed choices.

Further, as used herein, the term “comprising” or “including” is intended to mean that the methods, compositions, cells and kits includes the recited elements, but does not exclude others. Similarly, “consisting essentially of” is used to define methods, compositions, cells and kits that include the recited elements but exclude other elements that may have an essential significance on the functionality of the nucleic acid sequences, methods and populations of the inventions. “Consisting of” shall mean excluding other elements. Embodiments defined by each of these transition terms are within the scope of this invention.

Further, all numerical values, e.g., dose or ranges thereof, are approximations which are varied (+) or (−) by up to 20%, at times by up to 10%, from the stated values. It is to be understood, even if not always explicitly stated that all numerical designations are preceded by the term “about”. It also is to be understood, although not always explicitly stated, that the reagents described herein are merely exemplary and that equivalents of such are known in the art.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

### Example 1

### Construction of Suitable Expression Vectors for Functional Casein Proteins

The sequences of the αS1-casein, αS2-casein, β-casein and κ-casein genes were introduced into specific vectors for soybean expression, after codon optimization while maintaining their amino acid sequence. The nucleic acid sequences of these casein proteins (both original and optimized) are provided in Table 1 as well as the amino acid sequences in Table 2 below.

TABLE 1
Original and optimized nucleic acid sequences of bovine caseins
for expression in Soybean.

| | | | Nucleotide |
|---|---|---|---|
| | | | sequence, after |
| | | Original | codon |
| | Gene | nucleotide | optimization |
| Gene name | accession | sequence | for soybean |
| Alpha-S1-casein | CSN1S1 | SEQ ID NO: 1 | SEQ ID NO: 2 |
| (αS1) | | | |
| Alpha-S2-casein | CSN1S2 | SEQ ID NO: 3 | SEQ ID NO: 4 |
| (αS2) | | | |
| Beta-casein | CSN2 | SEQ ID NO: 5 | SEQ ID NO: 6 |
| (β) | | | |
| Kappa-casein | CSN3 | SEQ ID NO: 7 | SEQ ID NO: 8 |
| (κ) | | | |

TABLE 2
Amino acid sequence of bovine caseins for production in Soybean.

| Gene | Gene | |
|---|---|---|
| name | accession | Amino acid sequence |
| αS1 | CSN1S1 | SEQ ID NO: 9 |
| αS2 | CSN1S2 | SEQ ID NO: 10 |
| β | CSN2 | SEQ ID NO: 11 |

Alongside each bovine casein, various “helper proteins” were introduced that participate in the PTM process. It was further examined which one, if any, is needed. The examined “helper proteins” consisted of:
CSNK1: casein kinase 1
CSNK2: casein kinase 2

These “helper proteins” are bovine Casein kinases which underwent codon optimization to better suit soybean translation mechanisms, while the original amino acid sequence was maintained. The nucleic acid sequences of these helper proteins (both original and optimized) are provided in Table 3 as well as the amino acid sequences in Table 4 below.

TABLE 3
Original and optimized nucleic acid sequences of casein kinase
proteins (“helper proteins”) for expression in Soybean.

| | | Original | Nucleotide sequence, |
|---|---|---|---|
| | Gene | nucleotide | after codon |
| Gene name | accession | sequence | optimization for soybean |
| casein | CSNK1 | SEQ ID NO: 13 | SEQ ID NO: 14 |
| kinase 1 | | | |
| casein | CSNK2 | SEQ ID NO: 15 | SEQ ID NO: 16 |
| kinase 2 | | | |

TABLE 4
Amino acid sequence of casein kinase proteins for production in Soybean.

| Gene | Gene | |
|---|---|---|
| name | accession | Amino acid sequence |
| casein | CSNK1 | SEQ ID NO: 17 |
| kinase 1 | | |
| casein | CSNK2 | SEQ ID NO: 18 |
| kinase 2 | | |

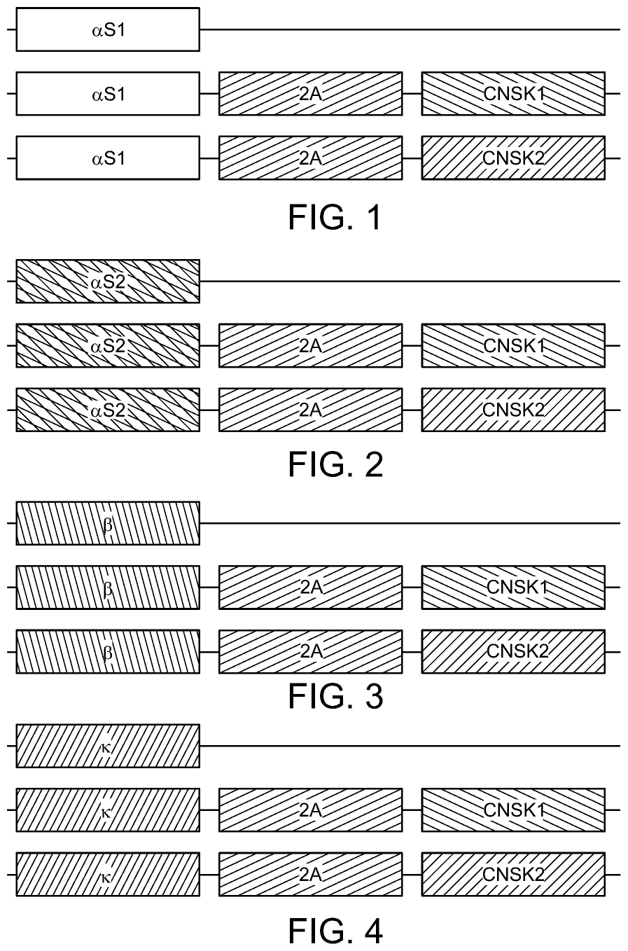

A total of 12 vectors were constructed as schematically represented in FIGS. 1 to 4: for each one of the four above mentioned casein proteins, three types of vectors were constructed and further tested: one comprising only the casein protein, one comprising the casein protein alongside with the casein kinase 1 protein (separated by a 2A self-cleaving peptide) and one comprising the casein protein alongside with the casein kinase 2 protein (separated by a 2A self-cleaving peptide). All the vectors were constructed under control of the p35S promoter and followed by the 35S terminator. The cloning procedure was performed using the GoldenGate molClo kit (Addgene, Watertown, Massachusetts, USA).

### Example 2

Expression of Suitable Vectors for Functional Production of Casein Proteins into Soybean Plant

Binary vectors were assembled using one of the above mentioned vectors alongside with pUBq-N7-NeonGreen-UBQ term as a visual selection marker, and introduced into Agrobacterium, strain K599. These were then used to induce hairy root in soybean seedlings. Protein from positive (NeonGreen expressing) roots were extracted and analyzed using Native-PAGE analysis, in which the proteins run as function of their structure rather than their size alone. This was followed by Western analysis using protein specific antibodies. Since the caseins are very similar and antibody's unspecific recognition is common, casein expression was separated one per plant, to avoid unspecific binding.

As a reference (positive control), commercially available bovine caseins were used. This analysis enabled to assess the 3D structure of the recombinant caseins from soybean hairy-roots.

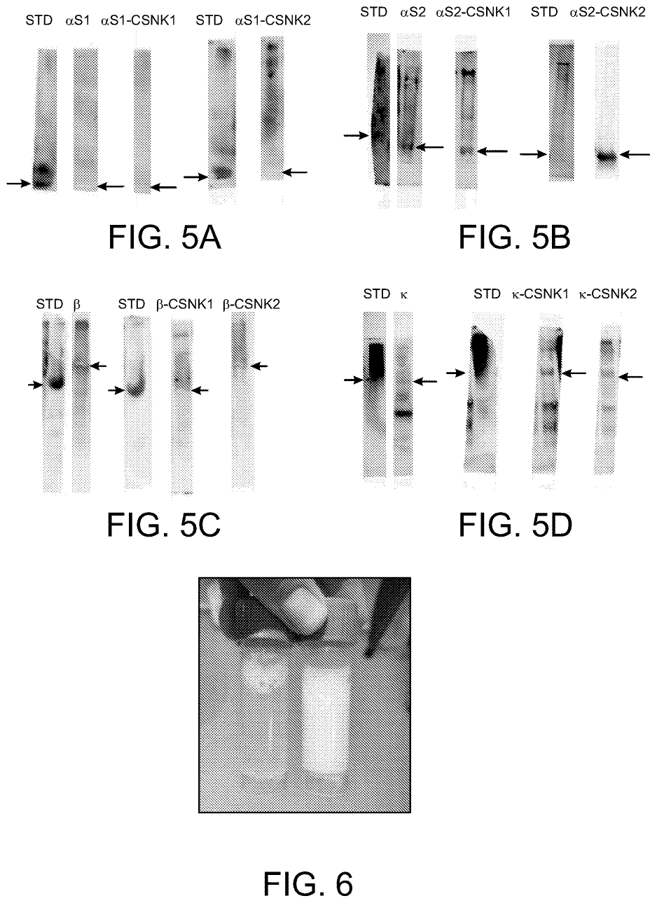

From the above experiments and as shown in FIG. 5, it appears that while αS1 and κ-caseins are folded correctly with soybean's natural PTM enzymes, αS2 and β-caseins require the CSNK2 and CNSK1 helper proteins for coherent folding, respectively.

### Example 3

### Verification of Post-Translation Modifications of Plant-Derived Bovine Caseins

To further verify the modifications that were produced on plant-derived caseins, MS analysis is performed in order to identify and characterize the various PTMs achieved on the recombinant caseins.

This analysis allows to point out what PTMs are naturally occurring in Soybean plant cells, and what PTMs are restored by the addition of helper proteins (CSNK1 or CSNK2).

### Example 4

### Micellization and Formation of Curd Formation Through Enzymatic Coagulation

Micellization protocol follows Knoop [3] and Akoi [4] using structured recombinant αS1-casein, αS2-casein, β-casein and κ-casein of Example 2 above.

The ratio between αS1:αS2:B:κ casein for micelle formation is 4:1:4:1 accordingly, and the concentration of the caseins in the Soy base is processed and targeted to 5%. All quantities of minerals and salt solutions are related to final casein micelles concentration of 2.5%.

TABLE 5
artificial casein formulations.

| | | | Volume for 10 ml | |
|---|---|---|---|---|
| | | | final suspension | |
| | | % In final | 1^{st} set of | 2^{nd} set of |
| Ingredient | Concentration | suspension | addition | addition |
| 4 Caseins mixture in | 5% | 50% | 5 ml | — |
| processed Soy base | | | | |
| CaCl₂ | 0.2M | 6.25% | 500 μl | 125 μl |
| K₂HPO₄ | 0.2M | 6.6% | 600 μl | 62.5 μl |
| K₃-citrate | 1M | 1% | 100 μl | — |
| DDW | | 36.1% | | 3.6 ml |

Recombinant caseins mixture is adjusted to pH-6.7 with 1M NaOH and heated to 37° C. All mineral solutions are adjusted to pH-6.7 and heated to 37° C.

A time interval of 15 min is taken between 1^{st} and 2^{nd} sets of additions.

After mixing all the ingredients the total suspension volume is adjusted to 10 ml with DDW and mixed for 2 hr for stabilization.

Light refraction of micelles is different than single and separated caseins. Therefore, a signal of successful micellization should be the appearance of the liquid—it should turn white and milky color as illustrated in FIG. 6.

The micelles shown in FIG. 6 are then subjected to enzymatic coagulation which is the first step of almost every cheese production. The enzymatic coagulation is conducted using RENNET (Chymosin) at 30-35° C., whereby the casein micelles lose their stability in the suspension and sediment as big curd particles.

Under the above conditions, Rennet cleaves κ casein that is located on the micelle surface in a specific site (between amino acids Phe105-Met106) and then casein micelles can interact with each other without k casein interruption.

In addition to temperature requirement, for best enzyme activity, the artificial milk proteins are subjected to slightly acidic pH and addition of Ca⁺² ions. For example, for Mozzarella production the pH is set at pH-6.3 and with external addition of Ca⁺² ions.

These conditions allow specific Ca+² solubility, to reach the desired balance between external and micellar ions.

### Example 5

### Characterization of Curd Protein

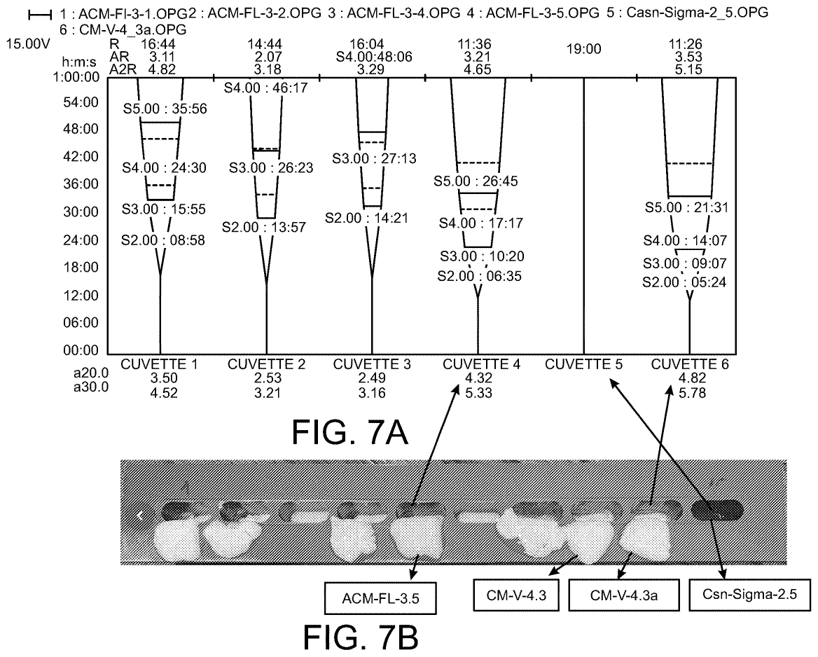

It is important to understand that not all micelles are functional micelles (i.e. have the ability to form curd). The effective/functional curding of artificial casein micelles is determined using two analytical tools:
Optigraph—A continuous analysis of NIR absorbance and transmission that allows the comparison of casein micelles enzymatic coagulation process (Start and development) time, and curd textural changes with time. The use of the system can be according to manufacture's instructions as detailed at “Optigraph—User's manual”. Alliance Instruments. Sep. 2004; 13-19
Texture analyzer—A texture analysis that uses a prob penetration technique with continuous force measurement many textural attributes can be analyze. The specific conditions for analysis can be obtained from manufacturer's manual such as “The texture analysis applications directory—Food products”. Stable micro systems. 2014; Issue 6, 4, 14.
Using Optigraph as an analytical tool for analyzing curding profile of caseins micelles, it is shown that the present micellization protocol (see FIG. 7: implemented in cuvette 4 on the Optigram (FIG. 7A) and in cuvette 5 in FIG. 7B, marked as trial ACM-FL-3.5) reveals similar profile as bovine casein micelles enzymatic curd (Curve marked as cuvette 6 (FIG. 7A) which belong to cuvette 9 in FIG. 7B)—Very close values of R (Time in min from adding RENNET to start coagulation), a20/30 and S2/3/4/5 (Time to reach certain gel strength or gel strength in a certain time) for cuvette 4 and bovine caseins micelles on cuvette 6.

With the same data (FIG. 7), it appears that other micellization protocols and singular casein suspension (curve of cuvette 5 (FIG. 7A) that belongs to cuvette 10 in FIG. 7B) give poor curd profile or no curd at all.

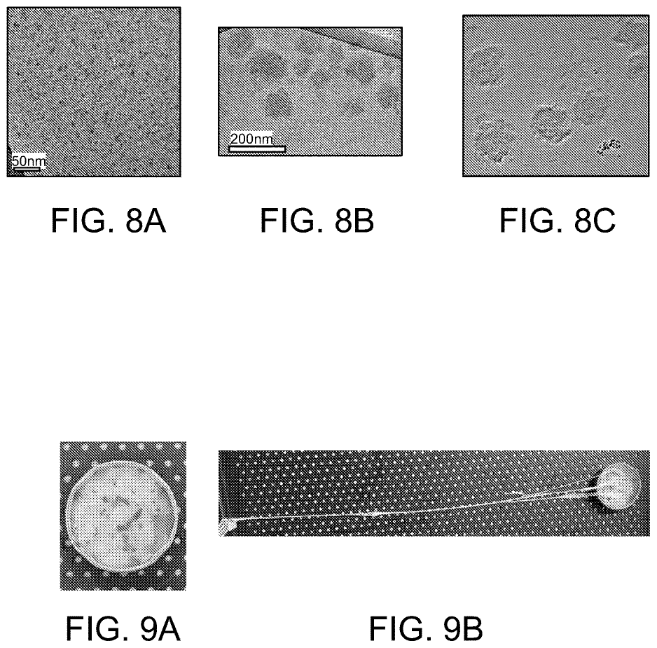

To further verify casein micelle structure, the resulting micelles were analyzed under TEM microscopy and were compared to bovine natural micelles (FIG. 8).

### Example 6

### Functional Micellization of Plant-Derived Caseins

In order to assess casein in vivo functionality, post translation modification in the plant, and their ability to form in vitro casein micelles, an in vitro protocol was first developed for singular bovine casein micellization. Briefly, micellization was performed according to the protocol described in Table 5, a protocol that results in suspension of stable caseins micelles with Ca, P and citrate concentration as in milk. During the micellization process, the casein solution is losing its transparency and becoming more turbid (FIG. 6).

To further verify that the recombinant soybean-produced caseins are sufficiently functional to form micelles, similar micellization process is repeated, using single recombinant caseins derived from transgenic soy. In one experiment, caseins are used with no helper protein and in the other, expression of both caseins and helper proteins is used, thus providing evidence for the necessity of the helper proteins in the production of active caseins.

The results are analyzed both by visual verifications (turbidity) as well as TEM observation.

Once soy-derived micelles are obtained, they are subjected to a similar Optigraph analysis, as described in Example 4, to verify that the micelles produced are actually active micelles and can form curd.

### Example 7

### Recombinant Curd Chemical Properties

Once a sufficient amount of recombinant caseins is obtained, they are assembled into micelles and subjected them to curding and mozzarella production process. The resulting cheese are then sent to composition analysis in order to test the content of sugars, salts (Na), proteins and carbohydrates.

### Example 8

### Preparation of Singular Caseins and Soy Based Cheese

To simulate the preparation of cheese of recombinant caseins from transgenic soy beans, a suspension made of 93% W/W Soybeans UF concentrate of MF filtrate (‘Soy base’ 1.5-2.0% soy proteins and carbohydrates), 2.3-2.4% W/W casein from bovine milk (C3400 Sigma-Aldrich), 1.5% W/W sugar and 3% vegetable fat is prepared. On first step, the micellization protocol described in Table 5 is used to create in vitro artificial casein micelles out of singular caseins in soy base. Then this “Hybrid” milk is homogenized and pasteurized and a ‘Process ready hybrid milk’ for cheese production is obtained. The milk is fermented with cheese culture (ChoozitTM81, Danisco) and the artificial in vitro casein micelles are coagulated using enzymatic coagulation (Maxiren 600, DSM). The curd is cut and squeezed into balls to release water for curd concentration. After fermentation, high quality curd of caseins is obtained for producing hard, semi hard, salted, pasta filata, soft, ripened cheese.

### Example 9

### Analysis of Cheese Properties (Melting, Stretching, Hardness Etc.)

A simple method of melting shredded Mozzarella cheese is used for analyzing cheese properties such as time for optimal melting, browning, length of stretching, appearance (shining surface), softness and fattiness of the texture etc. Few examples of physical analysis are described in Table 6 and FIG. 9.

The procedure of analyzing cheese properties is based on the main use of cheese as a layer on top a pizza dough. A simple protocol of specific weight—5 gr—of shredded cheese—0.5*0.5*2 cm—in oven on 250° C. The time takes to the sample to melt—to totally turn to soft texture, lose its initial shape but keep its shiny appearance, should be 3-5 min. The time taken for 20% of the surface area to become brown should be 7-10 min.

TABLE 6
Cheese physical properties.

| Parameter | | unit |
|---|---|---|
| Temperature | 250 | ° C. |
| Time for 20% (of surface) browning | 10 | min |
| Time for total melting | 5 | min |
| Stretching | >30 | cm |

1-56. (canceled)

57. A plant cell genetically modified to express at least one milk protein that is naturally expressed by a mammal and at least one kinase promoting at least one post translational phosphorylation in said expressed at least one milk protein, wherein said kinase is not FAM20C kinase.

58. The cell of claim 57, wherein at least one of:
(a) said at least one milk protein is coagulable;
(b) said at least one milk protein is a casein;
(c) said kinase is a casein kinase;
(d) said milk protein is one that is naturally produced by Bos taurus;
(e) said at least one milk protein is selected from the group consisting of αS1-casein, αS2-casein, β-casein and κ-casein, optionally wherein at least one of:
(i) said αS1-casein comprises an amino acid sequence as denoted by SEQ ID NO: 9, said αS2-casein comprises an amino acid sequence as denoted by SEQ ID NO: 10, said β-casein comprises an amino acid sequence as denoted by SEQ ID NO: 11 and/or said κ-casein comprises an amino acid sequence as denoted by SEQ ID NO: 12; and/or
(ii) said αS1-casein is encoded by a nucleic acid molecule comprising an nucleic acid sequence as denoted by SEQ ID NO: 2, said αS2-casein is encoded by a nucleic acid molecule comprising an nucleic acid sequence as denoted by SEQ ID NO: 4, said β-casein is encoded by a nucleic acid molecule comprising an nucleic acid sequence as denoted by SEQ ID NO: 6 and/or said κ-casein is encoded by a nucleic acid molecule comprising an nucleic acid sequence as denoted by SEQ ID NO: 8.
(f) said kinase is casein kinase 1 or casein kinase 2, optionally wherein at least one of:
(iii) said casein kinase 1 comprises an amino acid sequence as denoted by SEQ ID NO: 17 and/or said casein kinase 2 comprises an amino acid sequence as denoted by SEQ ID NO: 18; and/or
(iv) said casein kinase 1 is encoded by a nucleic acid molecule comprising a nucleic acid sequence as denoted by SEQ ID NO: 14 and/or said casein kinase 2 is encoded by a nucleic acid molecule comprising an nucleic acid sequence as denoted by SEQ ID NO: 16; and/or
(g) said milk protein is αs1-casein and said kinase is casein kinase 1 or casein kinase 2 and/or wherein said milk protein is β-casein and said kinase is casein kinase 1 or casein kinase 2.

59. A plant comprising at least one cell of claim 57, optionally being a genetically modified soybean.

60. A composition comprising at least one plant cell genetically modified to express at least one milk protein that is naturally expressed by a mammal and at least one kinase promoting at least one post translational phosphorylation in said expressed at least one milk protein, wherein said kinase is not FAM20C kinase.

61. The composition of claim 60, wherein at least one of:
(a) said composition comprising a portion of a plant comprising said plant cell, and/or
(b) said plant cell genetically modified to express at least one milk protein that is naturally expressed by a mammal and at least one kinase promoting at least one post translational phosphorylation in said expressed at least one milk protein, wherein said kinase is not FAM20C kinase.

62. A composition comprising a plant portion comprising at least one milk protein that is naturally produced by a mammal, wherein said milk protein is coagulable, wherein said plant portion comprises at least one cell of claim 57, or any harvested product, or tissue, or isolate, or extract, or secretion, or extrudate thereof.

63. The composition of claim 60, wherein at least one of:
(a) said composition is any one of a food, a medicament or a cosmetic;
(b) said composition is a micelle composition; and/or
(c) said composition is a dairy-like product, optionally wherein at least one of:
(i) said dairy-like product is selected from the group consisting of milk, cheese, curd, yogurt, cream and ice cream;
(ii) said dairy-like product is cheese, optionally
wherein texture or hardness or melt or stretch of the cheese is comparable to an animal-obtained dairy cheese or
wherein texture or hardness or melt or stretch of the cheese is improved compared to an animal-obtained dairy cheese.

64. A vector encoding for at least one milk protein that is naturally expressed by a mammal and at least one kinase promoting at least one post translational phosphorylation of said expressed milk protein, wherein said kinase is not FAM20C kinase.

65. The vector of claim 64, wherein at least one of:
(a) said at least one milk protein is a casein;
(b) said kinase is a casein kinase;
(c) said mammal is Bos taurus;
(d) said milk protein is at least one of αs1-casein, αs2-casein, β-casein and κ-casein, optionally wherein at least one of:
(i) said αS1-casein comprises an amino acid sequence as denoted by SEQ ID NO: 9, said αS2-casein comprises an amino acid sequence as denoted by SEQ ID NO: 10, said β-casein comprises an amino acid sequence as denoted by SEQ ID NO: 11 and said κ-casein comprises an amino acid sequence as denoted by SEQ ID NO: 12; and/or
(ii) said αS1-casein is encoded by a nucleic acid molecule comprising an nucleic acid sequence as denoted by SEQ ID NO: 2, said αS2-casein is encoded by a nucleic acid molecule comprising an nucleic acid sequence as denoted by SEQ ID NO: 4, said β-casein is encoded by a nucleic acid molecule comprising an nucleic acid sequence as denoted by SEQ ID NO: 6 and/or said κ-casein is encoded by a nucleic acid molecule comprising an nucleic acid sequence as denoted by SEQ ID NO: 8; and/or
(e) said kinase is of casein kinase 1 or casein kinase 2, optionally wherein at least one of:
(iii) said casein kinase 1 comprises an amino acid sequence as denoted by SEQ ID NO: 17 and/or said casein kinase 2 comprises an amino acid sequence as denoted by SEQ ID NO: 18; and/or
(iv) said casein kinase 1 is encoded by a nucleic acid molecule comprising an nucleic acid sequence as denoted by SEQ ID NO: 14 and/or said casein kinase 2 is encoded by a nucleic acid molecule comprising an nucleic acid sequence as denoted by SEQ ID NO: 16; and/or
(f) said milk protein is αS2-casein and said kinase is casein kinase 1 or casein kinase 2 and/or wherein said milk protein is β-casein said kinase is casein kinase 1 or casein kinase 2.

66. A plant cell comprising the vector of claim 64.

67. A plant comprising at least one cell of claim 66.

68. A method of producing a composition comprising at least one plant cell genetically modified to express at least one milk protein naturally expressed by a mammal and at least one kinase promoting at least one post translational phosphorylation in said expressed at least one milk protein, the method comprising:
a. providing at least one vector for expressing in a plant cell, at least one milk protein naturally expressed by a mammal and at least one kinase promoting at least one post translational phosphorylation in said milk protein, wherein said kinase is not FAM20C kinase;
b. transfecting at least one plant cell with said at least one vector;
c. providing conditions suitable for (i) expressing the at least one milk protein and the at least one kinase, and (ii) suitable for promoting said phosphorylation in said expressed milk protein.

69. The method of claim 68, wherein at least one of:
(a) said expressed at least one milk protein is coagulable;
(b) said vector is as defined in claim 64; and/or
(c) said plant is soybean.

70. A composition obtained by the method of claim 68.

71. A renneted curd composition obtained by the method of claim 68.

72. A dairy-like product obtained by the method of claim 68.

73. An artificially produced milk protein naturally expressed by a mammal obtained or obtainable from a plant source, wherein said milk protein is coagulable and wherein said protein was extracted from at least one cell as defined in claim 57, optionally said milk protein being a casein.

74. The milk protein of claim 73, wherein said casein is selected from the group consisting of αS1-casein, αS2-casein, β-casein and κ-casein.

75. The milk protein of claim 74, wherein said αS1-casein comprises an amino acid sequence as denoted by SEQ ID NO: 9, said αS2-casein comprises an amino acid sequence as denoted by SEQ ID NO: 10, said β-casein comprises an amino acid sequence as denoted by SEQ ID NO: 11 and said κ-casein comprises an amino acid sequence as denoted by SEQ ID NO: 12.

76. A plant part or portion comprising at least one milk protein that is coagulable, wherein said plant comprises at least one cell of claim 57.
